(19) United States
(12) Patent Application Publication
(10) Pub. No.: US 2025/0320550 A1
(43) Pub. Date: Oct. 16, 2025

(54) SEQUENCING BY SYNTHESIS USING ELECTROACTIVELY LABELED 3-OH-MODIFIED NUCLEOTIDES

(71) Applicant: Robert Bosch GmbH, Stuttgart (DE)

(72) Inventors: Nadezda FOMINA, Redwood City, CA (US); Armin DARVISH, Daly City, CA (US); Christopher JOHNSON, San Carlos, CA (US); Young Shik SHIN, Mountain View, CA (US); Gabrielle VUKASIN, San Jose, CA (US); Gary YAMA, Mountain View, CA (US); Christoph LANG, Sunnyvale, CA (US); Christian GRUMAZ, Waiblingen (DE); Sebastian PILSL, Rutesheim (DE)

(21) Appl. No.: 18/634,263

(22) Filed: Apr. 12, 2024

(51) Int. Cl. C12Q 1/6874; C12Q 1/48; C12Q 1/6825; C12Q 1/686

(52) U.S. Cl. CPC C12Q 1/6874; C12Q 1/485; C12Q 1/6825; C12Q 1/686; G01N 2333/9126

(57) ABSTRACT

Systems and methods for nucleic acid sequencing are provided. Polynucleotide strands to be sequenced are immobilized to a surface, clonally amplified and primed for polynucleotide synthesis. Nucleotides modified with cleavable electroactive labels at the 3′-OH of the sugar ring are provided and are incorporated into the growing strand during synthesis. The labels of nucleotides that are successfully incorporated into the growing polynucleotide strand are cleaved via application of a stimulus after incorporation and are detected by an electronic nanosensor. The systems and methods described herein combine the high accuracy of SbS methods combined with the scalability and speed of semiconductor-based electrical detection mechanisms.

### TECHNICAL FIELD

In at least one aspect, the present disclosure relates to systems, devices, and methods for nucleic acid sequencing.

### BACKGROUND

Many methods for sequencing polynucleotide strands are currently in use, all with benefits and drawbacks. Sequencing-by-synthesis (SbS) is currently the gold standard among currently employed polynucleotide sequencing methods, thanks to its high accuracy. It employs incorporation of nucleotides modified at the base with a fluorescent label and with a protecting group at the 3′-OH position. DNA sequence is determined by synthesizing a complimentary strand of DNA alongside the template strand by adding one modified nucleotide per synthesis cycle. Each cycle consists of incorporation of a modified nucleotide in a growing DNA strand, optical imaging to identify the type of nucleotide, and removal of labels and protecting groups. The methods' main limitation is long cycle time due to the need to optically image large flow cells. Accumulation of “scars” (parts of the linkers connecting labels to the bases that remain on the growing DNA strand after removal of the labels) leads to eventual termination of synthesis of complimentary strand, thus limiting the size of sequenced fragments to ˜150 bases. Other established sequencing methods offer longer read length and faster sequencing times.

### SUMMARY

In at least an aspect, a method for nucleic acid sequencing is provided. The method may comprise providing at least one device comprising an electronic nanosensor; providing a sample including a fragmented polynucleotide strand to the at least one device; clonally amplifying the fragmented polynucleotide strand within the at least one device to produce a clonally amplified cluster; exposing the clonally amplified cluster to a reaction solution comprising a polymerase enzyme capable of incorporating a nucleotide modified with an electroactive label covalently bound to the 3′-OH group of a sugar ring of the nucleotide into a polynucleotide strand, and at least one nucleotide modified with an electroactive label covalently bound to the 3′-OH group of a sugar ring of the nucleotide so that the at least one nucleotide modified with an electroactive label covalently bound to the 3′-OH group of a sugar ring of the nucleotide is incorporated into the polynucleotide strand; cleaving the electroactive label from the incorporated nucleotide so that the electroactive label diffuses toward the electronic nanosensor; and detecting a signal produced when the electroactive label is present within a sensing zone of the electronic nanosensor.

In another aspect, a system for nucleic acid sequencing is provided. The system may comprise at least one device. The at least one device may include at least one electronic nanosensor and a controller configured to provide a signal to the at least one device to promote delivery to the at least one device of a sample including a fragmented polynucleotide strand, and a reaction solution including a polymerase enzyme capable of incorporating a nucleotide modified with an electroactive label covalently bound to the 3′-OH group of a sugar ring of the nucleotide into a polynucleotide strand, and at least one nucleotide modified with an electroactive label covalently bound to the 3′-OH group of a sugar ring of the nucleotide. The controller may also be configured to provide a signal to the at least one device to promote clonal amplification of the polynucleotide strand and to promote incorporation of the at least one nucleotide modified with an electroactive label covalently bound to the 3′-OH group of a sugar ring of the nucleotide into the amplified polynucleotide strands. The controller may also be configured to apply a voltage to at least one electrode of the electronic nanosensor, and to measure the current (I) as a function of applied potential (V) when an electroactive label that has been cleaved from the incorporated nucleotide is present within the sensing zone of the electronic nanosensor.

In yet another aspect, a method for nucleic acid sequencing is provided. The method may comprise providing at least one device comprising an electronic sensor having a sensing electrode; providing a sample including a fragmented polynucleotide strand to the at least one device; clonally amplifying the fragmented polynucleotide strand within the at least one device to produce a clonally amplified cluster; exposing the clonally amplified cluster to a reaction solution comprising a polymerase enzyme capable of incorporating a nucleotide modified with an electroactive label covalently bound to the 3′-OH group of a sugar ring of the nucleotide into a polynucleotide strand, and at least one nucleotide modified with an electroactive label covalently bound to the 3′-OH group of a sugar ring of the nucleotide so that the at least one nucleotide modified with an electroactive label covalently bound to the 3′-OH group of a sugar ring of the nucleotide is incorporated into the polynucleotide strand; cleaving the electroactive label from the incorporated nucleotide so that the electroactive label diffuses toward the electronic sensor; applying a potential on the sensing electrode, wherein the potential oscillates between a reduction potential and an oxidation potential of the electroactive label; and detecting a signal transmitted with the oxidation and/or reduction of the cleaved electroactive label.

### BRIEF DESCRIPTION OF THE DRAWINGS

FIG. 1 illustrates systems and methods for sequencing nucleic acids according to an embodiment.

FIGS. 2A-C illustrate an electronic nanosensor including a microcavity according to an embodiment.

FIG. 3 illustrates an arrangement of a plurality of electronic nanosensors including a microcavity according to an embodiment.

FIG. 4 illustrates an electronic nanosensor including a microcavity sized to fit a magnetic bead according to an embodiment.

FIG. 5 illustrates structures of nucleotides labeled with electroactive molecules attached via a linker to the 3′-O position and chemistries to release the labels.

FIGS. 6A-6E illustrate examples of osmium electroactive labels with various functional groups according to at least some embodiments.

FIGS. 7A-7E illustrate examples of anthraquinone electroactive labels with various functional groups according to at least some embodiments.

FIGS. 8A-8G illustrate examples of ferrocene electroactive labels with various functional groups according to at least some embodiments.

FIGS. 9A-9G illustrate examples of phenothiazine electroactive labels with various functional groups according to at least some embodiments.

FIGS. 10A-10D illustrate examples of Methylene Blue electroactive labels with various functional groups according to at least some embodiments.

FIGS. 11A-11D illustrate examples of naphthalene 1,4-diol electroactive labels with various functional groups according to at least some embodiments.

FIGS. 12A-12E illustrate examples of catechol electroactive labels with various functional groups according to at least some embodiments.

FIG. 13 illustrates a method of synthesizing dNTPs labeled with example electroactive molecules attached via an allyl group at the 3′-OH position.

FIGS. 14A-14I illustrate examples of electroactive labels with alkyne functional groups.

FIG. 15 illustrates a method of synthesizing dNTP modified with an electroactive label attached via an azidomethyl group.

FIG. 16 illustrates a method of synthesizing dNTP modified with an electroactive label at the 3′-OH position via a disulfide group.

FIGS. 17A-17M illustrate examples of electroactive labels and linkers with carboxylic acid functional groups.

FIG. 18 illustrates a method of synthesizing dNTPs with electroactive labels attached at the 3′-OH position via an o-nitrobenzyl group.

FIG. 19 illustrates a method for synthesizing dNTPs with electroactive labels attached at the 3′-OH position via an amide group.

FIGS. 20A-M illustrate examples of electroactive labels and linkers with amine functional groups.

FIG. 21 illustrates an embodiment of the systems and methods of sequencing nucleic acids described herein using electroactive labels and electronic pH modulation.

FIGS. 22A and 22B illustrate a method for synthesizing dNTPs with electroactive labels attached at the 3′-OH position via an ester group.

FIGS. 23A-23K illustrate examples of electroactive labels and linkers.

FIG. 24 illustrates cyclic voltammograms of free ferrocene carboxylic acid and a ferrocene-labeled dTTP reversible terminator with an ester group and no linker.

FIG. 25 illustrates ferrocene carboxylic acid signal at different concentrations.

FIGS. 26A-C illustrate stability of a model dTTP 3′-OH ester.

FIG. 27 illustrates the results of a SPEX experiment in which incorporation of a dTTP-Fc ester was tested.

FIG. 28 illustrates a method for synthesizing a dTTP-Fc.

FIG. 29 illustrates voltammetry methods suitable for use with the systems and methods described according to at least an embodiment.

FIG. 30 illustrates suitable non-limiting examples of library preparation methods that may be used with the systems and methods for sequencing nucleic acids described herein.

FIG. 31 illustrates a concept for the readout circuit.

FIG. 32 illustrates the different phases for applying potentials to the electrode.

FIG. 33 illustrates different phases of redox status over time.

FIG. 34 illustrates the phase of attracting redox molecules electrostatically to the electrode surface which may be integrated with either the oxidation or the reduction phase.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about”. The first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

Unless indicated otherwise, all technical and scientific terms used herein have the same meaning as commonly understood by one of ordinary skill in the art to which the present disclosure belongs.

It is also to be understood that this disclosure is not limited to the specific embodiments and methods described below, as specific components and/or conditions may, of course, vary. Furthermore, the terminology used herein is used only for describing particular embodiments and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The terms “or” and “and” can be used interchangeably and can be understood to mean “and/or”.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

The terms “comprising”, “consisting of”, and “consisting essentially of” can be alternatively used. When one of these three terms is used, the presently disclosed and claimed subject matter can include the use of either of the other two terms.

The terms “polynucleotide”, “nucleotide”, “nucleotide sequence”, “nucleic acid”, “polynucleic acid”, and “oligonucleotide” are used interchangeably in this disclosure. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The terms “sequence identity” or “identity” refers to a specified percentage of residues in two nucleic acid or amino acid sequences that are identical when aligned for maximum correspondence over a specified comparison window, as measured by sequence comparison algorithms or by visual inspection. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity.

The term “comparison window” refers to a segment of at least about 20 contiguous positions in which a sequence may be compared to a reference sequence of the same number of contiguous positions after the two sequences are aligned optimally. In a refinement, the comparison window is from 15 to 30 contiguous positions in which a sequence may be compared to a reference sequence of the same number of contiguous positions after the two sequences are aligned optimally. In another refinement, the comparison window is usually from about 50 to about 200 contiguous positions in which a sequence may be compared to a reference sequence of the same number of contiguous positions after the two sequences are aligned optimally.

The terms “complementarity” or “complement” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 4, 5, and 6 out of 6 being 66.67%, 83.33%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 40%, 50%, 60%, 62.5%, 70%, 75%, 80%, 85%, 90%, 95%, 97%, 98%, 99%, or 100%, or percentages in between over a region of 4, 5, 6, 7, and 8 nucleotides, or refers to two nucleic acids that hybridize under stringent conditions.

Unless expressly stated to the contrary: all R groups (e.g. Rᵢ where i is an integer) include hydrogen, alkyl, lower alkyl, C₁₋₆ alkyl, C₆₋₁₀ aryl, C₆₋₁₀ heteroaryl, —NO₂, —NH₂, —N(R′R″)₂, —N(R′R″R′″)₃⁺L⁻, Cl, F, Br, —CF₃, —CCl₃, —CN, —SO₃H, —PO₃H₂, —COOH, —CO₂R′, —COR′, —CHO, —OH, —OR′, —O⁻M⁺, —SO₃⁻M⁺, —PO₃⁻M⁺, —COO⁻M⁺, —CF₂H, —CF₂R′, —CFH₂, and —CFR′R″ where R′, R″ and R′″ are C₁₋₁₀ alkyl or C₆₋₁₈ aryl groups; single letters (e.g., “n” or “o”) are 1, 2, 3, 4, or 5; in the compounds disclosed herein a CH bond can be substituted with alkyl, lower alkyl, C₁₋₆ alkyl, C₆₋₁₀ aryl, C₆₋₁₀ heteroaryl, —NO₂, —NH₂, —N(R′R″)₂, —N(R′R″R′″)₃⁺L⁻, Cl, F, Br, —CF₃, —CCl₃, —CN, —SO₃H, —PO₃H₂, —COOH, —CO₂R′, —COR′, —CHO, —OH, —OR′, —O⁻M⁺, —SO₃⁻M⁺, —PO₃⁻M⁺, —COO⁻M⁺, —CF₂H, —CF₂R′, —CFH₂, and —CFR′R″ where R′, R″ and R′″ are C₁₋₁₀ alkyl or C₆₋₁₈ aryl groups; the indication of a moiety or structure with positive charges implies that one or more negative counter ions are present to balance the charge, similarly, the indication of a moiety or structure with negative charges implies that one or more positive counter ions are present to balance the charge; percent, “parts of,” and ratio values are by weight; the term “polymer” includes “oligomer,” “copolymer,” “terpolymer,” and the like; molecular weights provided for any polymers refers to weight average molecular weight unless otherwise indicated; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

The term “alkyl” as used herein means C₁₋₂₀, linear, branched, rings, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Lower alkyl” refers to an alkyl group having 1 to about 8 carbon atoms (i.e., a C₁₋₈ alkyl), e.g., 1, 2, 3, 4, 5, 6, 7, or 8 carbon atoms. Lower alkyl can also refer to a range between any two numbers of carbon atoms listed above. “Higher alkyl” refers to an alkyl group having about 10 to about 20 carbon atoms, e.g., 10, 11, 12, 13, 14, 15, 16, 17, 18, 19, or 20 carbon atoms. Higher alkyl can also refer to a range between any two number of carbon atoms listed above.

The term “aryl” as used herein means an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether. Examples of aryl include, but are not limited to, phenyl, naphthyl, biphenyl, and diphenylether, and the like. Aryl groups include heteroaryl groups, wherein the aromatic ring or rings include a heteroatom (e.g., N, O, S, or Se). Exemplary heteroaryl groups include, but are not limited to, furanyl, pyridyl, pyrimidinyl, imidazoyl, benzimidazolyl, benzofuranyl, benzothiophenyl, quinolinyl, isoquinolinyl, thiophenyl, and the like. The aryl group can be optionally substituted (a “substituted aryl”) with one or more aryl group substituents, which can be the same or different, wherein “aryl group substituent” includes alkyl (saturated or unsaturated), substituted alkyl (e.g., haloalkyl and perhaloalkyl, such as but not limited to —CF₃), cycloalkyl, aryl, substituted aryl, aralkyl, halo, nitro, hydroxyl, acyl, carboxyl, alkoxyl (e.g., methoxy), aryloxyl, aralkyloxyl, thioalkyl, thioaryl, thioaralkyl, amino (e.g., aminoalkyl, aminodialkyl, aminoaryl, etc.), sulfonyl, and sulfinyl.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits. In the specific examples set forth herein, concentrations, temperature, and reaction conditions (e.g. pressure, pH, etc.) can be practiced with plus or minus 50 percent of the values indicated rounded to three significant figures. In a refinement, concentrations, temperature, and reaction conditions (e.g., pressure, pH, etc.) can be practiced with plus or minus 30 percent of the values indicated rounded to three significant figures of the value provided in the examples. In another refinement, concentrations, temperature, and reaction conditions (e.g., pH, etc.) can be practiced with plus or minus 10 percent of the values indicated rounded to three significant figures of the value provided in the examples.

In the examples set forth herein, concentrations, temperature, and reaction conditions (e.g., pressure, pH, flow rates, etc.) can be practiced with plus or minus 50 percent of the values indicated rounded to or truncated to two significant figures of the value provided in the examples. In a refinement, concentrations, temperature, and reaction conditions (e.g., pressure, pH, flow rates, etc.) can be practiced with plus or minus 30 percent of the values indicated rounded to or truncated to two significant figures of the value provided in the examples. In another refinement, concentrations, temperature, and reaction conditions (e.g., pressure, pH, flow rates, etc.) can be practiced with plus or minus 10 percent of the values indicated rounded to or truncated to two significant figures of the value provided in the examples.

In this application, electroactive molecules include Redox molecules. A Redox signal includes electrical signals such as a change in current. Polynucleic acid (NA) includes DNA, and nucleotides include dNTPs.

The processes, methods, or algorithms disclosed herein can be deliverable to/implemented by a processing device, controller, or computer, which can include any existing programmable electronic control unit or dedicated electronic control unit. Similarly, the processes, methods, or algorithms can be stored as data and instructions executable by a controller or computer in many forms including, but not limited to, information permanently stored on non-writable storage media such as ROM devices and information alterably stored on writeable storage media such as floppy disks, magnetic tapes, CDs, RAM devices, and other magnetic and optical media. The processes, methods, or algorithms can also be implemented in an executable software object. Alternatively, the processes, methods, or algorithms can be embodied in whole or in part using suitable hardware components, such as Application Specific Integrated Circuits (ASICs), Field-Programmable Gate Arrays (FPGAs), state machines, controllers or other hardware components or devices, or a combination of hardware, software and firmware components.

Throughout this application, where publications are referenced, the disclosures of these publications in their entireties are hereby incorporated by reference into this application to more fully describe the state of the art to which this invention pertains.

Sequencing-by-synthesis (SbS) is an acceptable standard among currently employed DNA sequencing methods, thanks to its high accuracy. It employs incorporation of nucleotides modified at the base with a fluorescent label and with a protecting group at the 3′-OH position. DNA sequence is determined by synthesizing a complimentary strand of DNA alongside the template strand by adding one modified nucleotide per synthesis cycle. Each cycle consists of incorporation of a modified nucleotide in a growing DNA strand, optical imaging to identify the type of nucleotide, and removal of labels and protecting groups. A limitation of the method is long cycle time due to the need to optically image large flow cells. Accumulation of “scars” (parts of the linkers connecting labels to the bases that remain on the growing DNA strand after removal of the labels) leads to eventual termination of synthesis of complimentary strand, thus limiting the size of sequenced fragments to ˜150 bases. Other established sequencing methods offer longer read length and faster sequencing times.

Pacific Biosciences of California, Inc. of Menlo Park, California has a single-molecule real time (SMRT) sequencing technology that uses an enzyme immobilized to the surface of a zero-mode waveguide (ZMW). When a sequencing reaction begins, the enzyme incorporates nucleotides modified with fluorophores at the 5′-OH group into the growing DNA chain. A high-resolution camera of the ZMW records the fluorescence of the successive nucleotides being incorporated in a movie-like fashion. After each nucleotide incorporation, the fluorophore is released from the growing DNA chain by the action of the enzyme which leaves behind no “scars”, so the next nucleotide with a fluorophore can come in. When utilizing a particular library preparation technique, the same circular DNA fragment can be read multiple times, thus overcoming the high error rates associated with real-time sequencing.

Semiconductor sequencing technologies can give rise to affordable and rapid benchtop sequencing systems. Technologies such as the Ion Torrent sequencing technology from ThermoFisher Scientific of Waltham, Massachusetts for example use an array of semiconductor chips that detect nucleotide incorporation events by sensing small changes in pH. Such technologies require no specialized enzymes and no modification of native nucleotides.

Nanopore sequencing technologies such as that developed by Oxford Nanopore Technologies PLC of Oxford, United Kingdom, for example, record changes in current through a biological nanopore that result from nucleic acids passing through the nanopore. Each of the nucleotides dATP, dGTP, dCTP, and dTTP/dUTP has a unique current modulation signature. Each type of nucleotide can thus be identified without using any labels. Such approaches offer rapid sequencing of long DNA fragments but lack single base resolution or sufficient accuracy for clinically relevant applications. Additionally, 1.5 million nucleotides can translocate through a nanopore per second. Reading each nucleotide therefore requires sampling rates higher than 3 MHz and a detection limit of a few pA, which is challenging and costly to build from an electronics perspective.

Redox cycling is an electrochemical technique used for the ultrasensitive detection of electroactive molecules down to the level of a single molecule. For example, many immunosensors used to detect antibody-antigen interactions have been reported based on this technique. The sensing performance of redox cycling devices is determined by the device geometry. Signal amplification increases the longer an electroactive molecule resides between the reducing and oxidizing electrode. The amplification factor scales inversely with the distance between reducing and oxidizing electrodes. For instance, a molecule shuttles faster between two closely situated electrodes than between electrodes that are farther away from each other. The amplification factor scales directly with the surface area of the electrodes in that a molecule has more chances to interact with larger surfaces. The distance between the electrodes has been limited to tens to hundreds of nanometers due to fabrication limitations. Thus, most sensors reported to date use both interdigitated electrodes and nano-slits to maximize the surface area of the electrodes exposed to the molecules.

A number of nucleotide reversible terminators with different 3′-O-blocking chemical groups have been reported. Such nucleotide reversible terminators have also been incorporated in commercial products for SbS. For example, allyl, azidomethyl, (2-aminoethoxy)-3-propionyl, tert-butyldithiomethyl, amino, or 2-nitrobenzyl groups have been incorporated in commercial SbS systems However, none of these systems have utilized 3′-reversible terminators incorporating electroactive labels.

Provided herein are systems and methods for sequencing polynucleic acids. Polynucleotide strands to be sequenced are immobilized to a surface and primed for polynucleotide synthesis. Nucleotides modified with cleavable electroactive labels at the 3′-OH are provided and are incorporated into the growing strand during synthesis. The labels of nucleotides that are successfully incorporated into the growing polynucleotide strand are cleaved after incorporation and are detected by an electrochemical nanoelectrode sensor. The systems and methods described herein therefore provide a combination of the high accuracy of SbS methods combined with the scalability and speed of semiconductor-based electrical detection mechanisms.

The electrochemical nanoelectrode sensor, similar to that described in US 2019/0137435 A1, may include two electrodes separated by a nanoscale thick dielectric layer. The electrodes may each be held at a different voltage to enable electron transfer via an electroactive label. The small space between the two electrodes, the dielectric layer, may function as a sensing zone. The width of the sensing zone is defined by the thickness of the dielectric layer. The electrochemical nanoelectrode sensor may also be referred to herein as a nanogap sensor or an electronic nanosensor.

Electroactive labels include moieties capable of electron transfer in a circuit upon the application of an electric field. Non-limiting examples of suitable electroactive molecules that may be used as labels include organometallic complexes (e.g., ferrocene and its derivatives, osmium and ruthenium complexes), organic molecules (e.g., tetrathiafulvalene, methylene blue, anthraquinone, phenothiazine, aminophenol, nitrophenol, erythrosine B, ATTO MB2), and metal nanoparticles. The electroactive species must be capable of electron transfer under applied electrical potential to produce current signal.

Each type of nucleotide (dATP, dCTP, dGTP, dTTP/dUTP) may be modified with a label having a unique current-voltage (I-V) signature. The type of nucleotide added to a growing polynucleotide strand may thus be identified based on the current-voltage (I-V) characteristics of a signal detected when the particular nucleotide is incorporated. This cycle may be repeated until the entire DNA sequence may be reconstructed. Alternatively, only one type of label can be used. In that case the sample can be split into multiple channels and only one (unique) type of nucleotide is modified in each channel. The sequencing data from multiple channels will need to be aligned in order to reconstruct the entire sequence.

In an embodiment, primers, polymerase, buffer salts, and up to four types of 3′-O-labeled dNTPs (dATP, dCTP, dTTP/dUTP, and dGTP) may be added to the system. After a suitable dNTP is incorporated, the extension process may stop since 3′-O labeled dNTPs act as reversible terminators of the DNA polymerization process as described in U.S. Pat. No. 7,541,444B2. Enzymes and unincorporated dNTPs may then be washed away, and a chemical reagent may be added that may cleave the label and exposing the 3′-OH for the addition of the next dNTP. The cleaved electroactive labels may diffuse towards the sensor where the signal may be detected in the form of a current. The labels may be used to provide a unique electronic signature for each type of nucleotide and to facilitate their identification. To identify an incorporated nucleotide, a controller may apply voltages to the electronic nanosensor in the form of, for example, potentiometry, cyclic voltammetry, square wave voltammetry, or linear sweep voltammetry. The applied voltages may cover a pre-set range corresponding to redox reactions of the electroactive labels. The controller may measure the current (I) as a function of applied potential (V). The signature of each electroactive label may be determined prior to utilizing the electroactive label in a sequencing reaction. As each electroactive label has a unique I-V signature, this unique signature may be used to identify the label, and therefore the nucleotide carrying the label. Voltages may be swept on both electrodes on a nanogap sensor independently (applying reducing voltages to the first electrode and oxidizing voltages to the second electrode). Alternatively, a wave form that covers both reducing and oxidizing potentials may be applied to one or both electrodes.

Conventional SbS methods may suffer from diminished signal and lower accuracy after several cycles of extension which limits the overall length of polynucleotide fragments that can be read to ˜150 bases. This limitation may be due to a number of factors including dephasing of the clonal clusters and/or the presence of extra organic moieties that remain attached to the nucleobases (which may be referred to as “scars”) which accumulate on the growing DNA strand with each cleavage cycle. According to various embodiments, nucleotides may be modified with electroactive labels using cleavable linkers that leave no scars upon removal of the label, promoting the read out of longer segments of polynucleotide strands with high accuracy. The electronic readout provided in various embodiments is also faster compared to classical fluorescent readout, as electronic sensors may be arranged in an array where multiple sites are read in parallel.

Optical sequencing techniques rely on detecting multiple optical labels usually distinguished by the wavelength of the emitting light. This often requires different excitation illumination and optical filter sets and a lot of mechanical moving parts to achieve multi-color detection. The instruments are thus bulky and expensive, and the sequencing workflow is slow. Electrical detection in sequencing methods such as that utilized by Oxford Nanopore sequencing relies on resolving very fast events of single nucleotides interacting with the nanoscale cavity of a biological nanopore. Due to challenges stemming from the speed of such measurements, the accuracy is limited. The systems and methods described herein improve on both systems by using a polymerase enzyme combined with electrochemical detection. Since electrochemical detection of distinct redox labels may be achieved by simply changing applied electrical potential and measuring the current, the systems and methods of one or more embodiments described herein may detect multiple nucleotides at very high speed with no moving parts with compact and relatively cost-effective electrical instruments. For instance, sampling frequencies on the order of tens to hundreds of kilohertz may be easily achievable on handheld instruments, making it possible to record a real-time snapshot of enzyme activities with a time resolution hundreds of times faster than expensive cameras. For example, in the context of many enzymes working in tandem, distinct signals from enzymes that are out of sync can be expected since a polymerase enzyme incorporates new base-pairs at a rate of several nucleotides per second, which may be hundreds of times slower than average electrochemical sampling frequencies. Therefore, the systems and methods described herein improve on the speed, read length and accuracy of current optical techniques. Additionally, because the systems and methods described herein use average ensemble detection of many enzymes working in tandem, they may not suffer from the limitations of nanopore sequencing, where fast detection of single molecules are required. The stochastic nature of single-molecule events in the case of nanopore sequencing makes it hard to read each nucleotide with high confidence. This difficulty results in a high error rate. While these errors can be mitigated by using expensive computers and complex computational algorithms, such an approach will add to the bulkiness and cost as well as to the overall time-to-result of nanopore sequencing. Such an approach is therefore not suitable for applications where cost and speed are of paramount importance (e.g., sequencing at point-of-care or at home). The sequences and methods described in one or more embodiments herein, therefore, improve on all the available sequencing platforms by combining the advantages of sequencing by synthesis with that of fast electrochemical detection.

Therefore, one or more embodiments enable a sequencing platform that is both fast and affordable for applications at point-of-care. For example, the high frequency of signal detection in one or more embodiments improves on one of the major challenges in current sequencing platforms where dephasing of enzymes working in parallel limits the accuracy and read length of the sequencing platforms. Higher frequency electrical detection may resolve enzyme activity at a much shorter timescale, hence distinguishing between incorporation activities by separate out-of-sync enzymes.

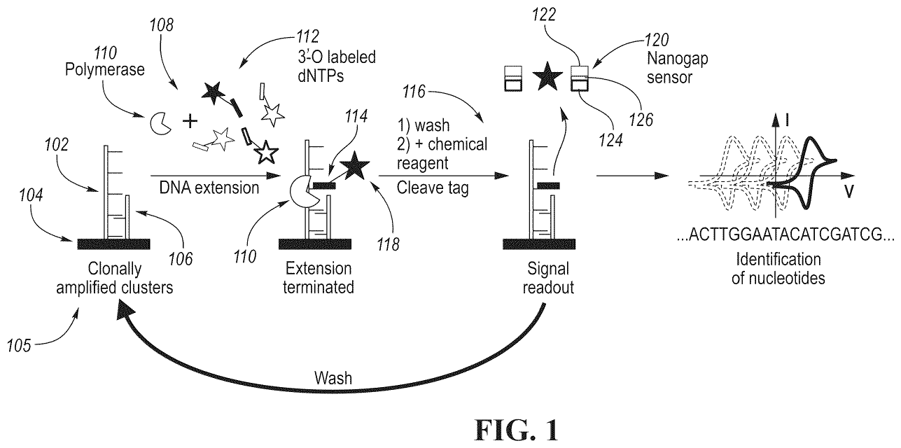

FIG. 1 illustrates a method for sequencing polynucleic acids according to an embodiment. A sample including polynucleotide strands to be sequenced may be subjected to fragmentation to break the polynucleotide strands into strands of a length that may reasonably be sequenced, fragmented strands 102. The fragmented strands 102 may then be adhered to a surface 104. The fragmented strands 102 may then each be replicated to form clusters of fragments having the same sequence (clonally amplified clusters 105). The terms clonally amplified clusters, clusters, clonal amplicons, and clones all refer to the clusters of polynucleotide strands formed by a replicated fragmented strand 102 of a unique sequence. The fragments may be clonally amplified into clusters 105 using bridge amplification as described in U.S. Pat. No. 7,115,400B1. The surface 104 may alternatively be in the form of a bead and clusters 105 may be formed by using emulsion PCR on beads as described in US20050079510A1. The final structure may be in the form of the surface 104 to be closely positioned to the nanogap sensor 120. The polynucleotide strands of the clonally amplified clusters 105 may bind to their complementary primers 106 and they may be exposed to a reaction mix 108. The reaction mix 108 may be a solution. The reaction mix 108 may include a polymerase enzyme 110 capable of incorporating nucleotides modified with an electroactive label on the 3′-OH group 112. The reaction mix 108 may also contain nucleotides modified with an electroactive label on the 3′-OH group 112. The reaction mix 108 may include all four types of nucleotides (dATP, dCTP, dGTP, and/or dTTP/dUTP). Each type of nucleotide may be modified with an electrochemically distinct label. Alternatively, the reaction mix 108 may include only one type of nucleotide (e.g., dATP), or the reaction mix 108 may include two types of nucleotides, or the reaction mix 108 may include three types of nucleotides. In addition to the polymerase enzyme 110 and modified nucleotides 112, the reaction mix may contain buffers and other additives that may be useful for promoting the sequencing reaction. Using the primed polynucleotide strands of the clonally amplified clusters 105 as a template, the polymerase enzyme 110 may incorporate a complementary dNTP 114 into the polynucleotide strand to be sequenced. Because the electroactive label is covalently bound to the 3′-OH group of the dNTP, the reaction is terminated. Unincorporated labeled dNTPs 112 from the reaction mix 108 may then be washed away. The strands being sequenced 105 may then be exposed to a stimulus 116 that modulates cleavage of the electroactive label 118 from the incorporated dNTP 114, exposing the 3′-OH group for a subsequent round of incorporation. The cleaved label 118 may then be free to diffuse toward the nanogap sensor 120, and the signal from the cleaved label 118 may be detected in the form of a current. The nanogap sensor may include a first electrode 122, a second electrode 124, and a dielectric layer 126 defining a sensing zone between the first electrode 122 and the second electrode 124. After the signal from the cleaved label 118 is detected, the label may be washed away and another round of incorporation may begin.

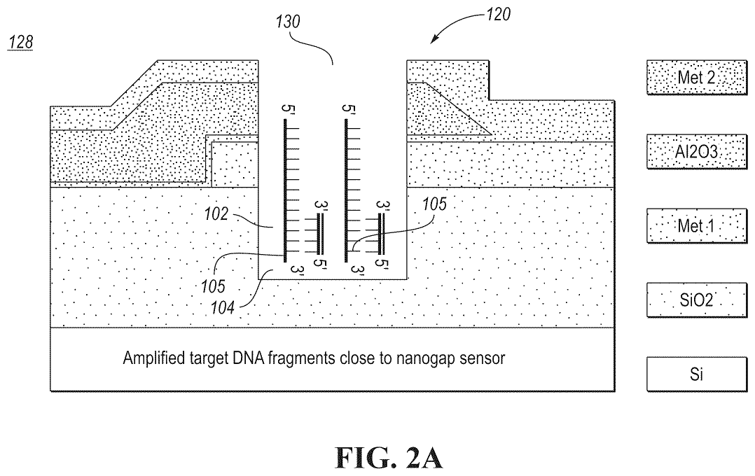

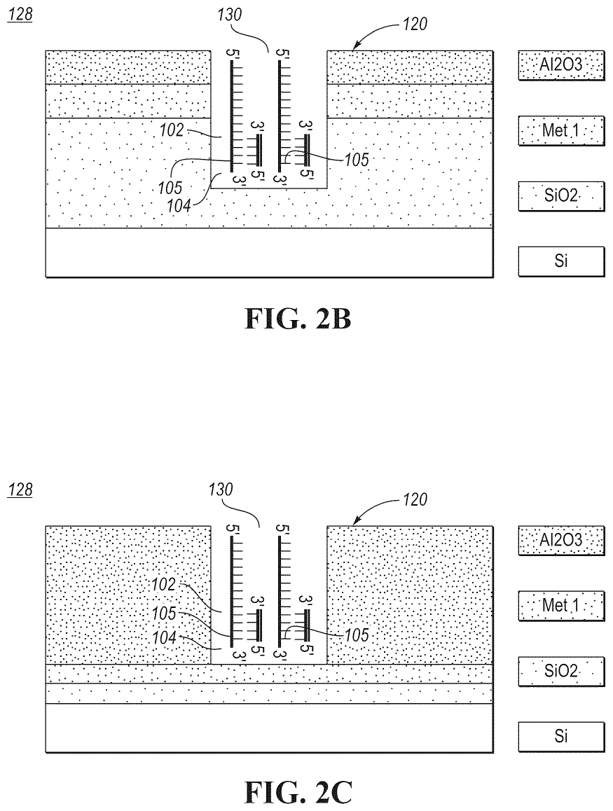

Referring to FIG. 2A, a device 128 may be provided having a plurality of microcavities 130 according to an embodiment. In an example, an electronic nanosensor 120 may be integrated in each microcavity 130. The electronic nanosensor may include two electrodes. Each microcavity 130 may contain a cluster of clonal amplicons 105. The clonal amplicons 105 may be introduced to the microcavity 130 by adhering the fragmented polynucleotide strands 102 to the walls of a microcavity with each microcavity containing only one fragmented sequence. The fragmented sequence may then be clonally amplified by bridge-PCR along the walls of the microcavity 130. FIGS. 2B and 2C illustrate examples of electronic nanosensors having one electrode instead of two.

The cluster of clonal amplicons may be immobilized on the surface of a bead that may fit into a microcavity. A fragmented polynucleotide strand 102 may be adhered to the surface of a bead through chemical linkage or hybridization to primers, with each bead containing only one sequence. Clonal amplification may then take place on the beads. Adhering the clone clusters 105 to beads may enable multiple loadings of a single chip. Additionally, this clonal bead-based reloading strategy may enable higher throughput in sequence reads on a single chip with a limited number of sensors 120 and microcavities 130 since the same sensors 120 may be reused to sequence more fragments. The beads may be formed from materials including but not limited to sepharose, polystyrene, magnetites and/or functional polymers. The beads may be formed from magnetic materials that promote easy manipulation. For example, magnetic beads may improve the efficiency of loading and unloading the microcavities.

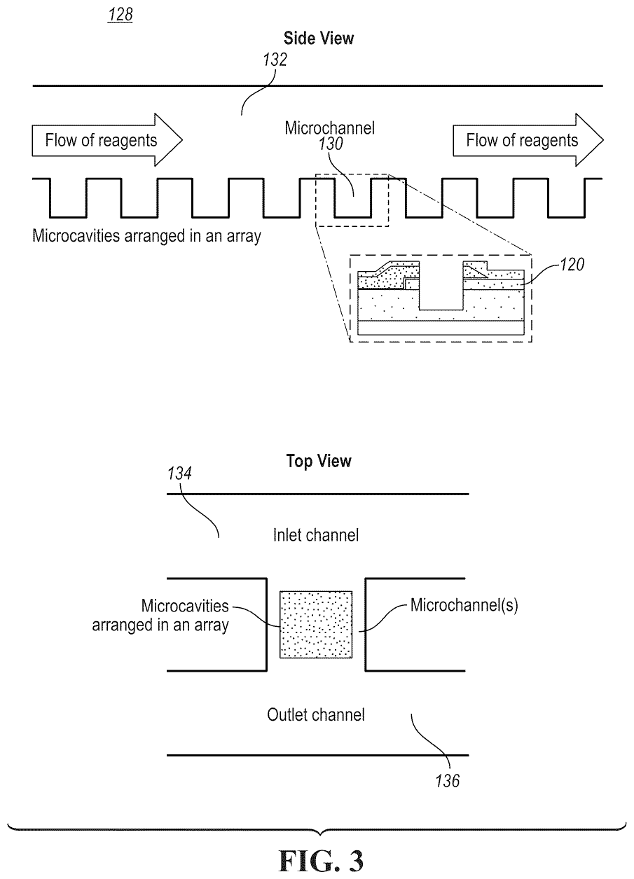

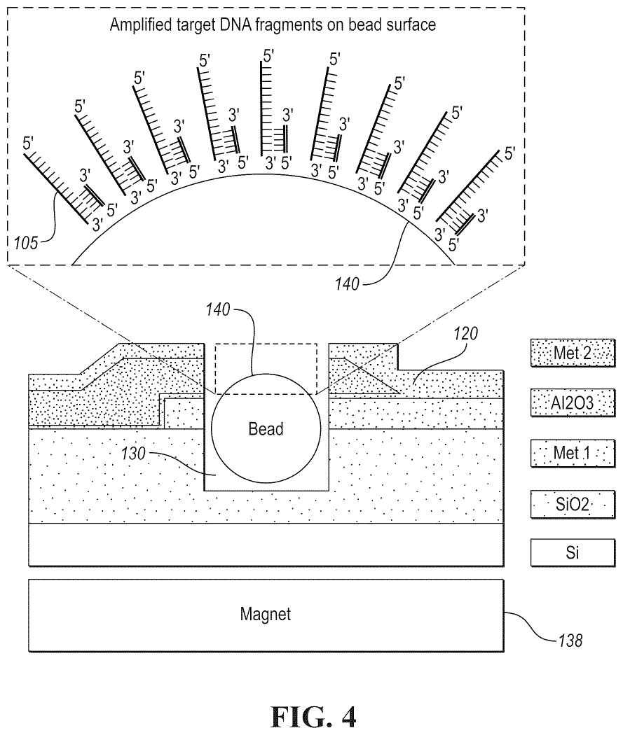

As described in FIG. 2A, the device 128 may include a plurality of microcavities 130 each including an electronic nanosensor 120. The microcavities 130 may also be referred to as wells. The microcavities or wells 130 may be arranged in an array of individually addressable electronic nanosensors 120. The array may be fabricated on a Complementary Metal-Oxide Semiconductor (CMOS) chip for example. The chip may operate as a controller. The chip may interface with the external environment via microfluidics to allow flow of the components. In an example, as depicted in FIG. 3, the device 128 may include an array of electronic nanosensors 120 with microcavities 130. Each microcavity 130 may be in fluid communication with a microchannel 132. Fluid solutions such as the reaction mix 108 may be flowed over the microcavities 130 via the microchannel 132. The device 128 may include additional channels such as an inlet channel 134 and an outlet channel 136 to regulate the flow of solutions through the device. The device 128 may also include a magnet 138. The magnet 138 may be in magnetic communication with a magnetic bead 140 in a microcavity 130. The clonally amplified clusters 105 produced by clonal amplification of fragmented nucleotide strands 102 may be adhered to the surface of the bead 104 as depicted in FIG. 4. The magnet 138 may be used to manipulate the placement of the bead 140 in the well. In this way, the controller may operate to regulate the flow of the sample and reaction components. The controller may also regulate the clonal amplification and sequencing reactions by altering the flow of components and/or the environmental conditions within the device including but not limited to temperature, pH, or light.

As described above, the signals generated by the electroactive labels may be detected by an electronic nanosensor. Briefly, the sensor may include a first electrode and a second electrode separated by a nanoscale thick dielectric layer. The first and second electrodes may be held at different voltages to enable electron transfer via the electroactive label. The small space between the two electrodes (the dielectric layer) may act as a sensing zone. The width of the sensing zone is thus defined by the thickness of the dielectric layer. In an embodiment, the sensing zone may be small enough to generate an amplified signal through redox cycling amplification, where an electroactive molecule undergoes an electrochemical reaction (for example, oxidation) on the first electrode, then diffuses to the second electrode where it undergoes the opposite reaction (reduction). The molecule may diffuse back and forth between the first and second electrodes resulting in an amplified electrical signal. In another embodiment, the thickness of the dielectric layer is on the same order as the size of the electroactive molecule itself, such that the molecule interacts with both electrodes simultaneously and completes the electrical circuit. In this embodiment, while the electroactive molecules reside in the sensing zone, electrons may transfer between the two electrodes, producing an amplified current signal per electroactive molecule. This signal may be much higher than a signal that would be expected from a single electron transfer event. This mechanism of signal generation may be a limiting case of redox shuttling. Another mechanism of signal generation that may occur in a nanogap sensor such as the nanogap sensor described herein is electron tunneling through an electroactive molecule. Unlike redox cycling, tunneling does not involve structural changes, such as generation of charge, change of redox state, addition or loss of atoms, or rearrangement of covalent bonds within the electroactive molecules. Instead, the label acts as a bridge between two electrodes that allows for the flow of electrons. Regardless of the mechanism, the electric current generated by the sensor may be robust enough to sense and identify each nucleotide in a polynucleotide sequence.

Each type of dNTP may be modified with a unique electroactive label having redox properties that are distinct from other labels used in a set. For example, dATP may be modified with electroactive label 1; dGTP may be modified with electroactive label 2; dCTP may be modified with electroactive label 3; and dTTP may be modified with electroactive label 4. All four dNTPs may be added to a single reaction together with the polymerase enzyme and other additives including but not limited to salts, Mg²⁺, or co-factors. For example, a reaction may also include dimethyl sulfoxide (DMSO), formamide, or detergents to increase template accessibility; bovine serum albumin (BSA) to prevent adherence of the polynucleotide strands to walls; or polyethylene glycol (PEG) or glycerol to increase reaction specificity. At each DNA extension cycle the electronic nanosensor may produce a signal with a unique signature corresponding to electroactive label 1, 2, 3, or 4. In this way, each subsequent nucleotide incorporated may be identified as dATP, dGTP, dCTP, or dTTP/dUTP, respectively. In another embodiment the same label may be used to modify all four types of dNTPs. In each incorporation cycle only one type of labeled dNTP may be added to the reaction. In this case the electronic nanosensor may produce a signal only when a dNTP complementary to the fragmented polynucleotide strand is added.

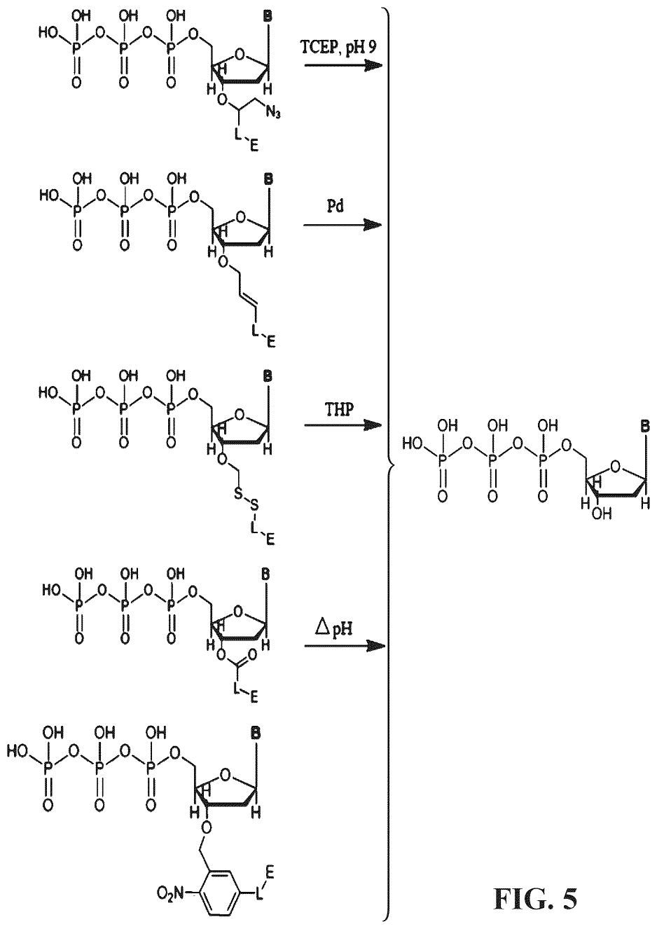

FIG. 5 illustrates examples of how electroactive molecules may be attached to the nucleotides. The labels may be attached to the 3′-OH position of the nucleotides via a linker and a cleavable functional group. Non-limiting examples of cleavable functional groups include groups commonly employed in SBS research, such as azidomethyl, allyl, or disulfate groups. Other cleavable groups include esters, nitrobenzyl, silyl, methoxymethyl, and other groups as described in Wuts, P., “Greene's protecting groups in organic synthesis”, John Wiley &Sons, 2014. Azidomethyl groups may be removed with tris(2-carboxyethyl)phosphine (TCEP). Allyl groups may be removed with Pd or a Pd complex including Pd(PPh3)44). Disulfate groups may be removed with trihydroxypropylphosphine (THP). The cleavable group must be stable under polynucleotide strand extension conditions and must be capable of being selectively removed to expose the 3′-OH end of the growing DNA chain to allow for subsequent addition of the next nucleotide.

Non-limiting examples of linkers include a hydrocarbon chain which may contain heteroatoms such as O, N, and S. Examples of modifying nucleotides at the 3′-OH position with fluorophores and other functional moieties have been described. Similar synthetic strategies may be employed to attach an electroactive molecule to the 3′-OH position.

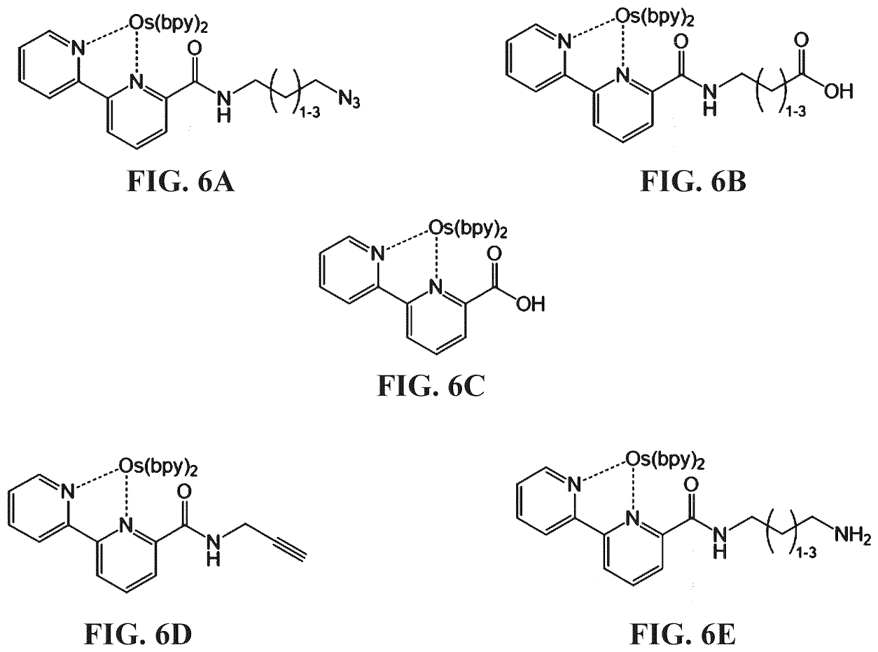

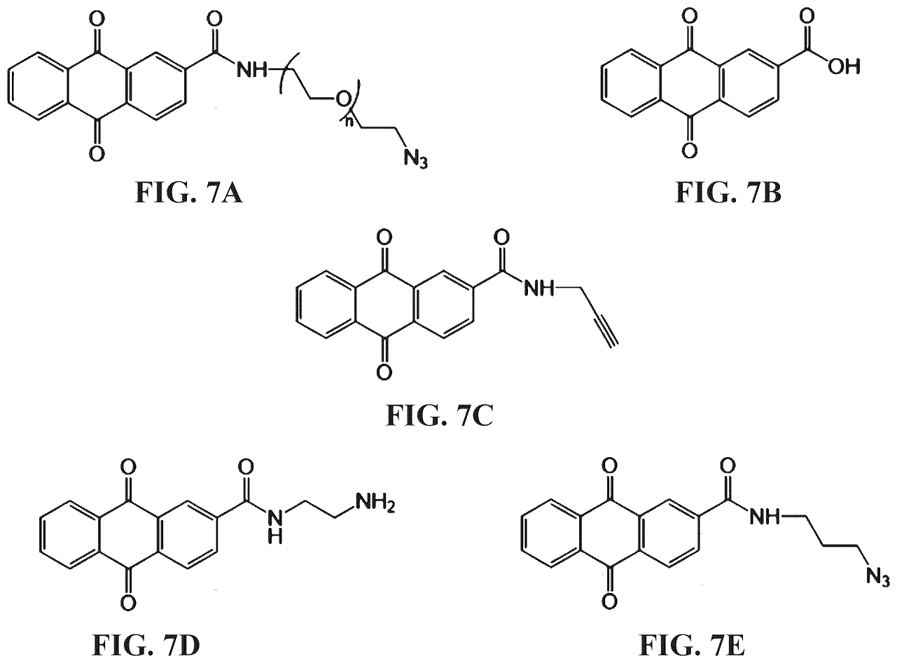

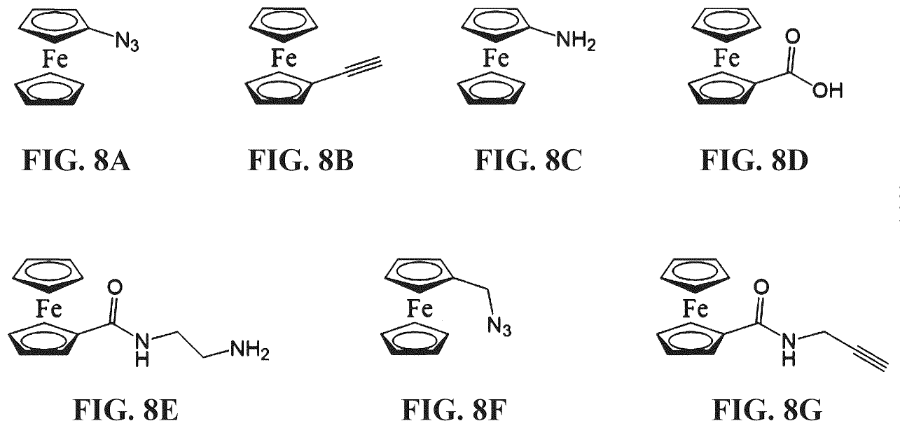

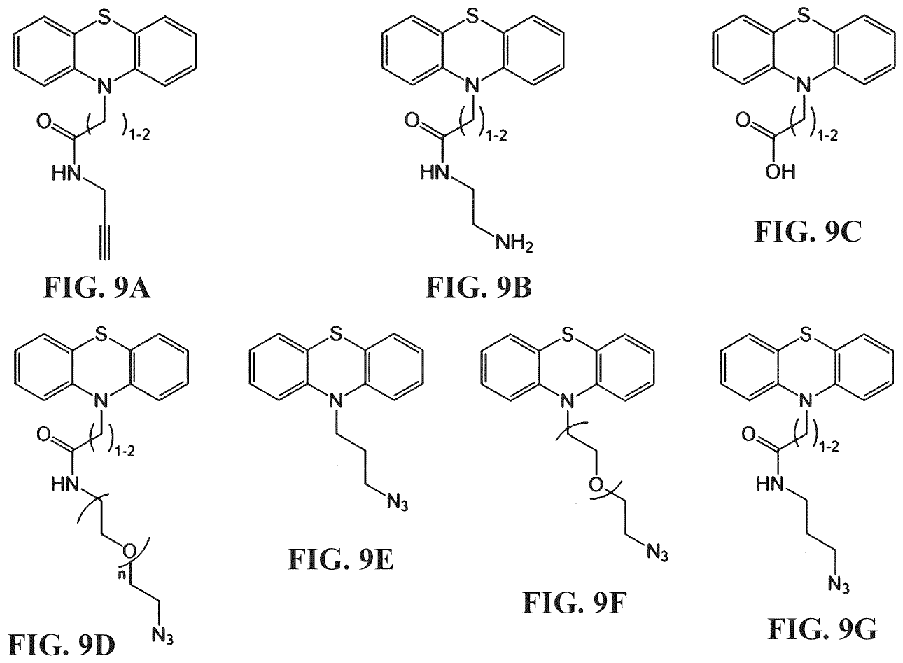

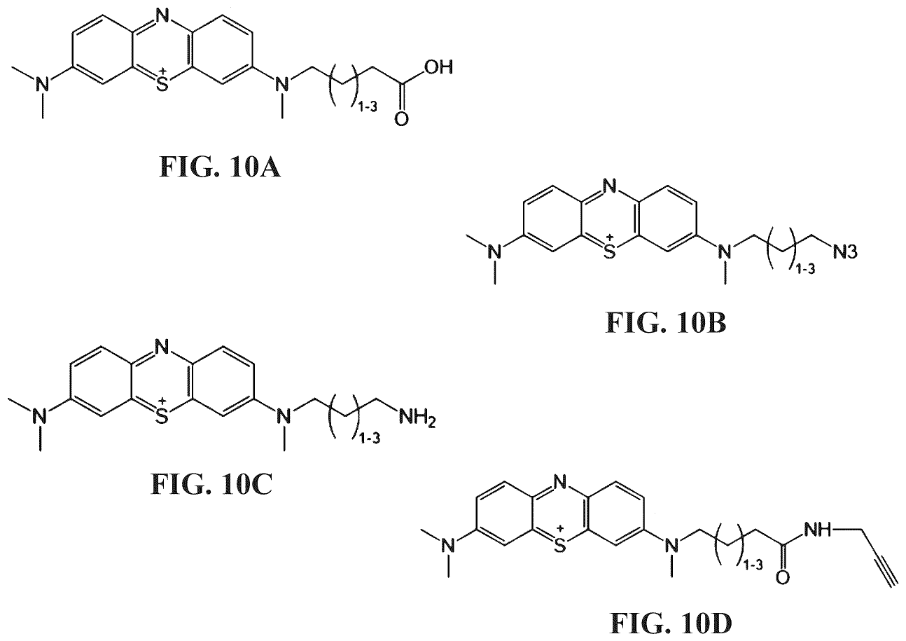

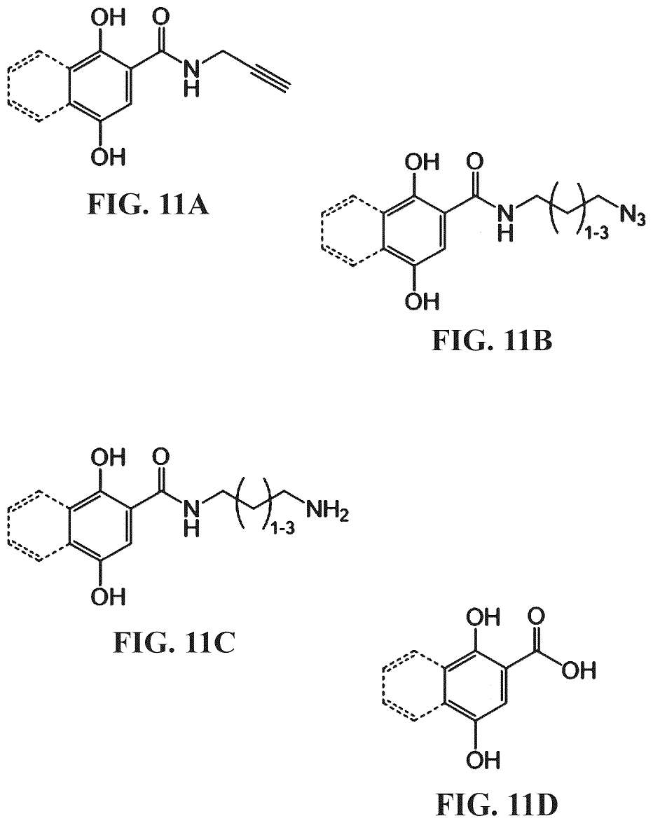

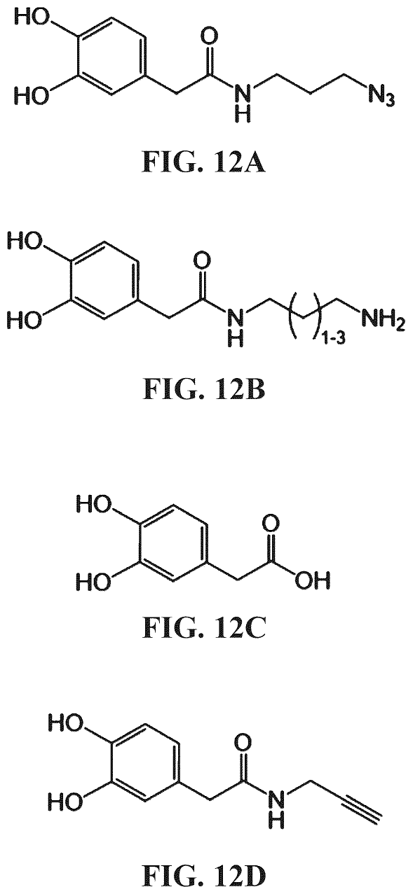

Examples of electroactive labels with various functional groups suitable for attachment to dNTPs via a variety of linkers and chemistries are shown in FIGS. 6-12. For example, FIGS. 6A-6E illustrate structures for example osmium electroactive labels with or without a linker. FIGS. 7A-7E illustrate structures for example anthraquinone electroactive labels with or without a linker. FIGS. 8A-8G illustrate structures for example ferrocene electroactive labels. FIGS. 9A-9G illustrate structures for example phenothiazine electroactive labels. FIGS. 10A-10D illustrate structures for example methylene blue electroactive labels. FIGS. 11A-11D illustrate structures for example naphthalene 1,4-diol electroactive labels. FIGS. 12A-12D illustrate structures for example catechol electroactive labels.

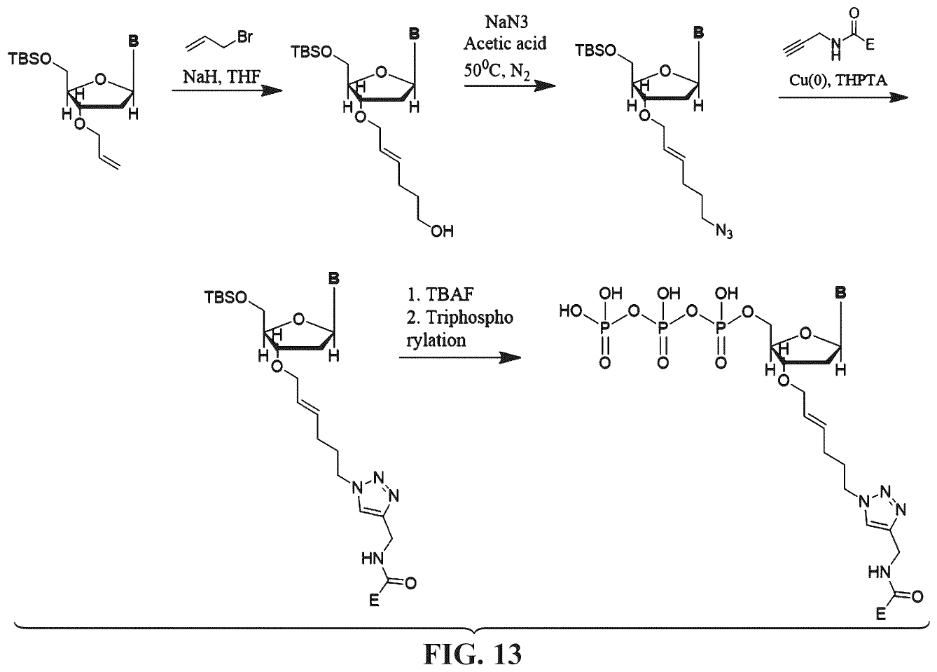

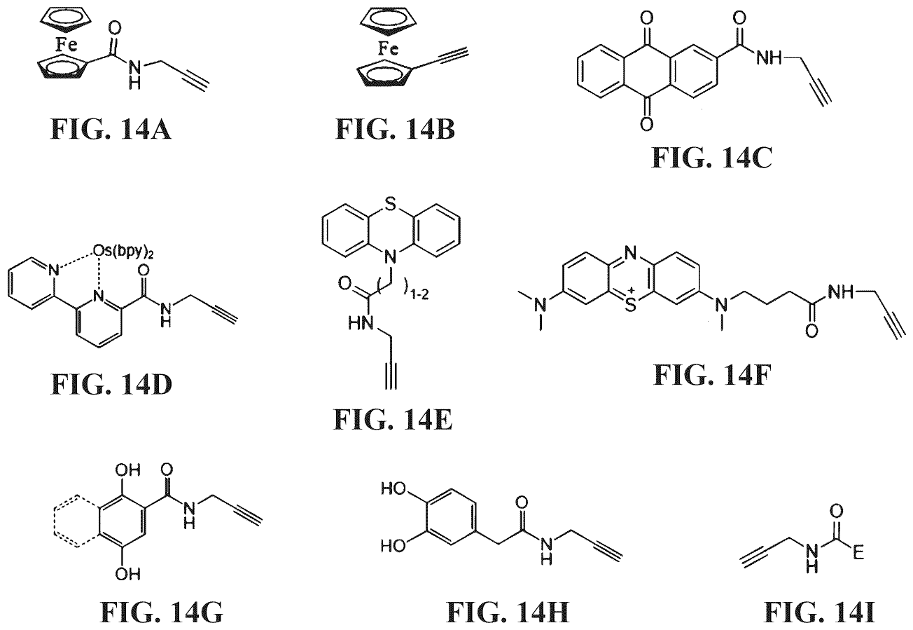

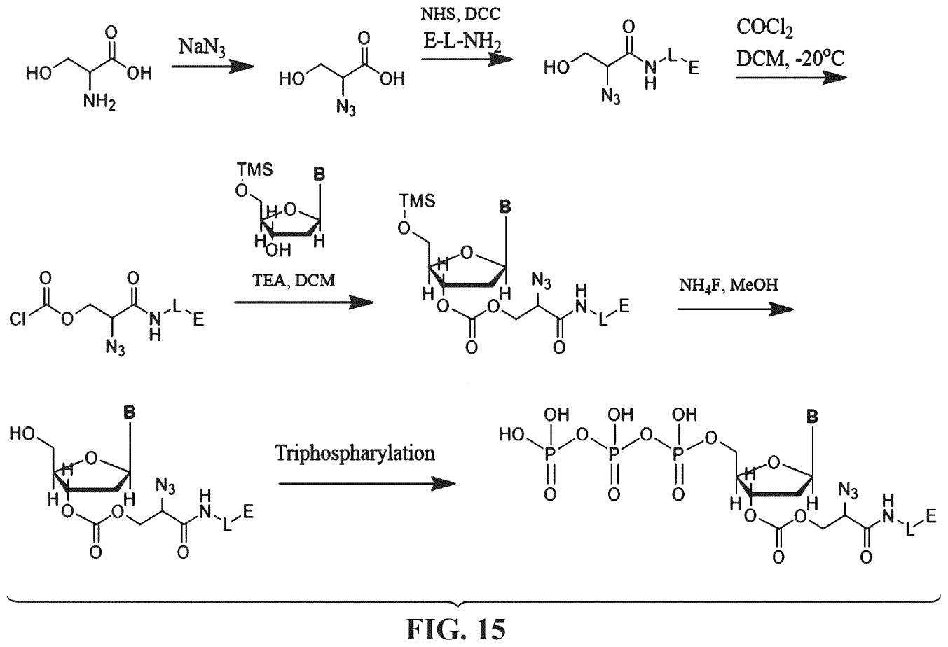

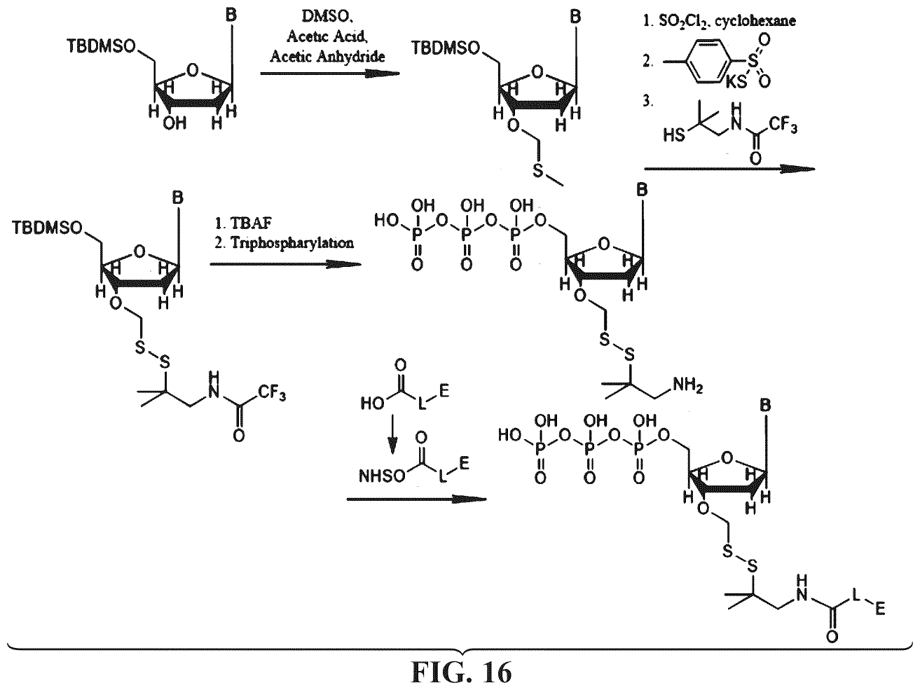

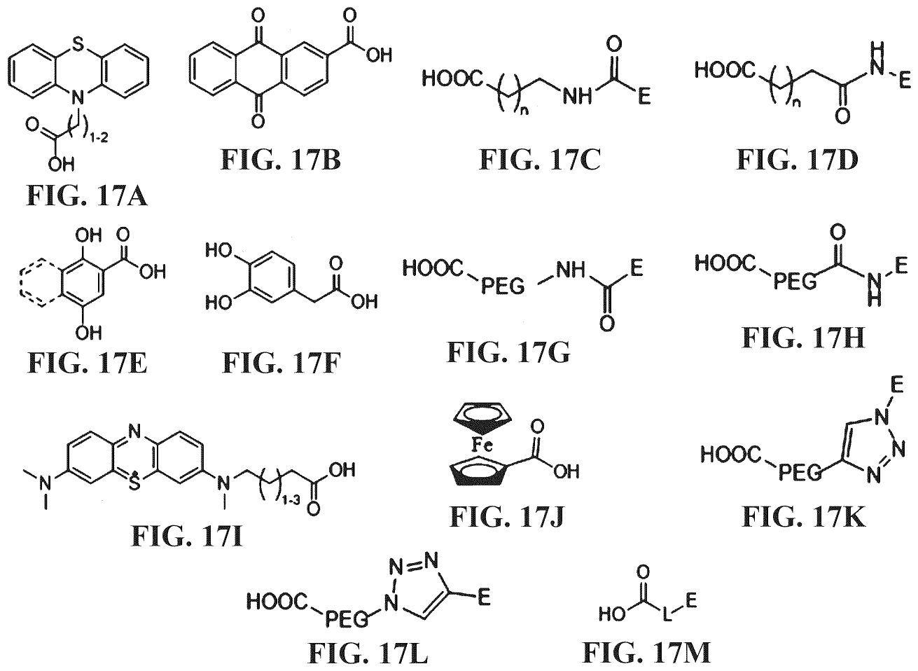

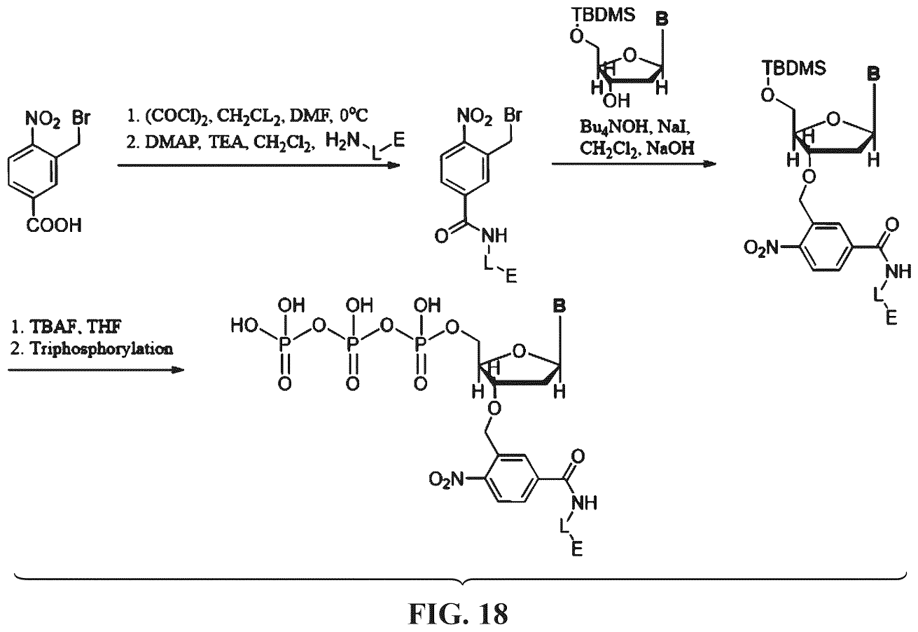

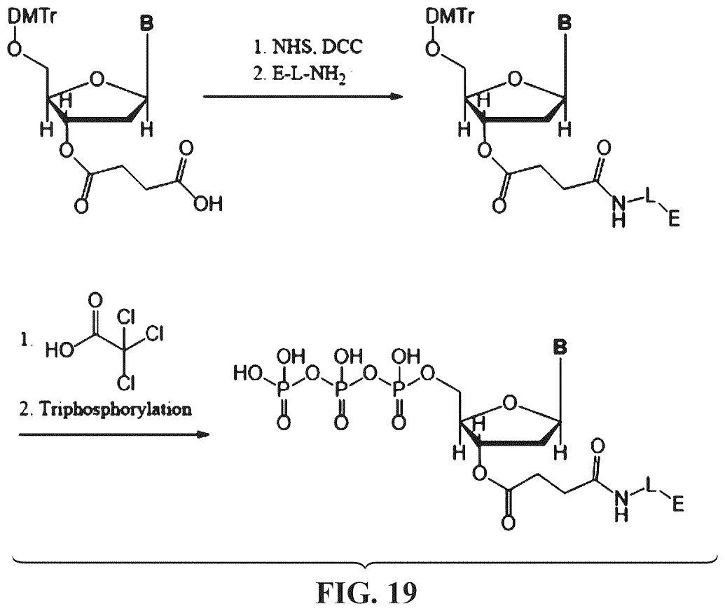

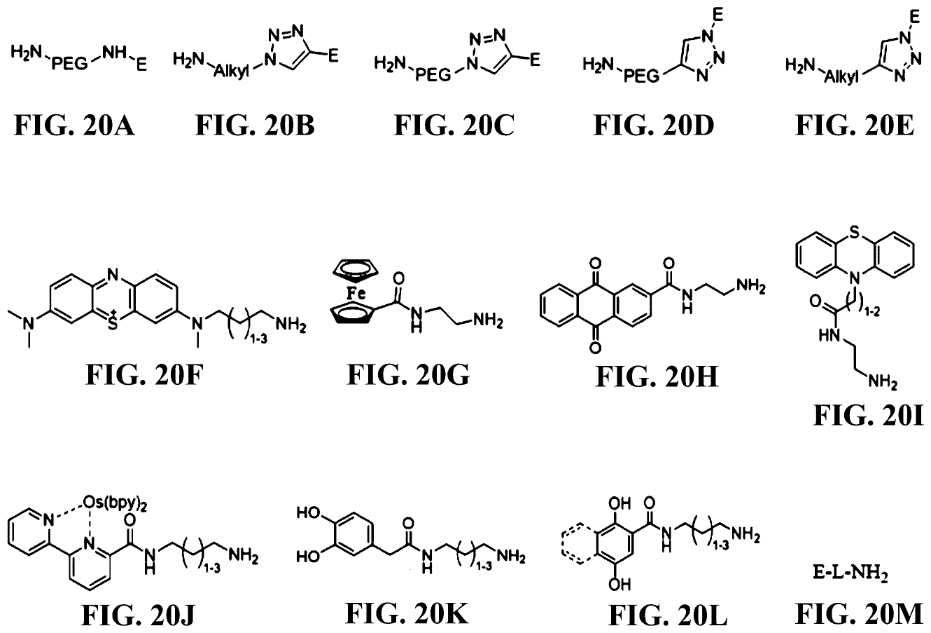

FIG. 13 illustrates a synthetic route for synthesizing nucleotides with electroactive labels attached at the 3′-OH position via an allyl group. FIGS. 14A-14H illustrate examples of electroactive labels with terminal alkyne functional groups. The general formula for these examples is shown in FIG. 14I, where E refers to the electroactive label. FIG. 15 illustrates a synthetic route for synthesizing dNTPs modified with an electroactive label attached via an azidomethyl group. FIG. 16 illustrates a synthetic route for synthesizing dNTPs modified with an electroactive label at the 3′-OH position via a disulfide group. FIGS. 17A-17F illustrate examples of electroactive labels with carboxylic acid functional groups. FIGS. 17G-17L illustrate examples of suitable linkers that may be utilized with carboxylic acid functional groups. FIG. 17M illustrates a general formula for an electroactive label with a linker and carboxylic acid functional group, where E refers to the electroactive label, and L refers to the linker. FIG. 18 illustrates a synthetic route for synthesizing dNTPs with electroactive labels attached at the 3′-OH position via an o-nitrobenzyl group. FIG. 19 illustrates an example route for synthesizing electroactive labels with functional amide groups. FIGS. 20A-20E illustrate examples of suitable linkers that may be utilized with amine functional groups. FIGS. 20F-20L illustrate examples of electroactive labels with amine functional groups. FIG. 20M illustrates a general formula for an electroactive label with a linker and an amine functional group, where E refers to the electroactive label, and L refers to the linker.

In various embodiments, the nucleotide modified with an electroactive label on the 3′-OH group 112 may have the following formula:

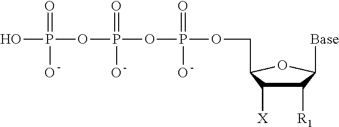

wherein:
X is

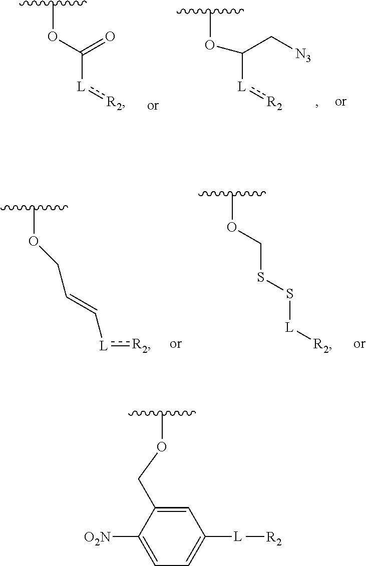

[symbol] is a single bond, a double bond, a triple bond,
Base is Adenine (A), Cytosine (C), Guanine (G), Thymine (T), or Uracil (U), L (Linker) is absent or a hydrocarbon chain comprising between 1 and 1000 atoms which may contain heteroatoms such as O, N, and S,
n=1-1000
R₁ is H or OH, and
R₂ is a redox label.

An example of a nucleotide modified with an electroactive label on the 3′-OH group 112 includes a compound having the following formula:

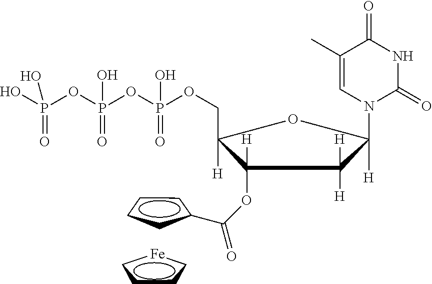

As described in FIG. 1, after a complementary nucleotide modified with an electroactive label at the 3′-OH position 112 has been incorporated, the polynucleotide strand being sequenced may be exposed to a stimulus that modulates cleavage of the electroactive label from the incorporated dNTP, exposing the 3′-OH group for a subsequent round of incorporation. The cleaved label may then be free to diffuse toward the electronic nanosensor, and the signal from the cleaved label may be detected in the form of a current. The stimulus may be a chemical reagent that must be added to the system at every cycle. An external physical trigger may also be used to cleave the electroactive label and unmask the 3′-OH group. The use of a physical trigger may further streamline the sequencing process. For instance, the use of a physical trigger may be advantageous as it may eliminate the need for washing steps or reduce the number of required reagents. Additionally, a physical trigger is not flow dependent and is orthogonal to other chemical steps of the process. In this way, use of an external physical trigger may allow for a more compact and fast sequencing instrument suitable for point of care use. Non-limiting examples of external physical triggers include light, electric current or voltage, temperature, and/or pH. Each of these triggers may be utilized alone, or combinations of two or more triggers may also be utilized.

Examples of photocleavable groups have been utilized in biomedical technologies, such as drug delivery and gene sequencing. Additionally, ortho-nitrobenzyl has been widely employed as a UV light-sensitive protecting group for biomolecules.

An ester group or an acetal group may also be employed as a cleavable moiety in combination with electric current. Incorporation of (2-aminoethoxy)-3-propionyl dNTPs into a growing DNA strand by polymerases of the A-family group has been described. The 3′-ester moiety is hydrolyzed by the polymerase during incorporation. This hydrolysis is slow, however under conditions of neutral pH. As ester hydrolysis is a pH-dependent process, external pH control may be applied to drive cleavage of the ester group and subsequent release of an electroactive label on demand. To prevent spontaneous or premature ester hydrolysis, polymerases without esterase function may be utilized for incorporation of labeled nucleotides. Alternatively, the pH may be kept at neutral or slightly acidic levels until it is time to trigger the release of redox labels. In the case of an acetal group, hydrolysis will occur in acidic pH, while it will be stable at neutral or slightly basic pH suitable for incorporation by a polymerase. Technology promoting fast pH manipulation in solution using electric current is described in US20140274760A1, US20220018806A1, U.S. Ser. No. 10/942,146B2, and US20200363371A1. Briefly, electric current may be applied to an electrode in contact with a solution containing an additive which undergoes electrochemical oxidation or reduction to produce or consume H+. This reaction may result in a local change of pH in the vicinity of the electrode. During polynucleotide strand sequencing such as during the sequencing of DNA, the pH should remain between pH 4 and pH 9. Depurination and phosphodiester bond breakage may occur at pH levels below 4. Furthermore, denaturation of the DNA can occur at pH levels above 9.

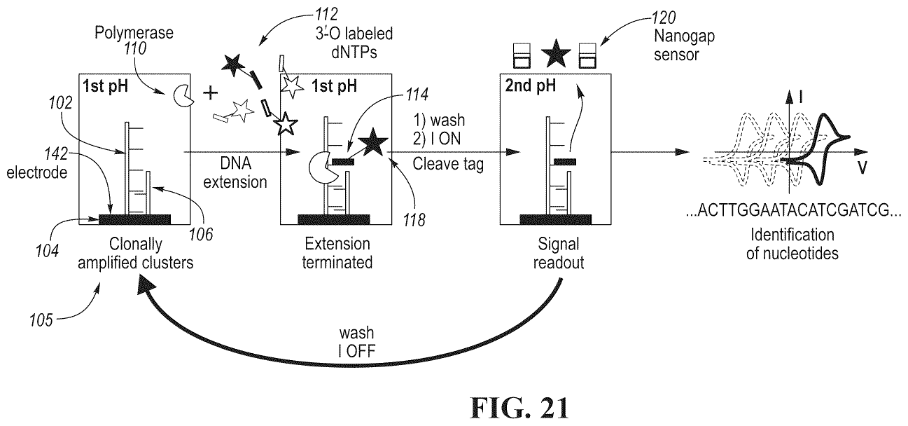

FIG. 21 illustrates how electronic pH modulation may be combined with pH-sensitive cleavable groups, such as ester and acetal groups, to enable polynucleotide strand sequencing such as DNA sequencing. Using electrochemical pH modulation technology, an optimal pH (1st pH) may be maintained at the step corresponding to incorporation of the modified dNTP 112 into a growing polynucleotide strand. Once incorporation is complete, the pH may be changed electronically to a 2nd pH by applying a current to a working electrode 142. The working electrode 142 may comprise the surface to which the clonally amplified clusters 105 are adhered 104 or may be close enough to the clonally amplified clusters 105 to regulate the pH locally near the clusters 105. The new pH value may be optimal for the removal of a 3′-O-protecting group, for example, through ester hydrolysis. Therefore, the electroactive label may be removed, and the 3′-end of the DNA may become available for incorporation of the next nucleotide. Other pH-sensitive chemical groups may also be utilized in a similar manner.

Where the electroactive label is attached to the 3′-OH position via an ester functional group, the cleavage process may be regulated by a combination of pH and temperature. For example, to enhance the cleavage process, a polymerase of interest may be fused to a pH-dependent esterase which is also temperature-controlled. The ester hydrolysis may therefore be accelerated at beneficial pH and temperature (e.g., 40° C.) after washing residual nucleotides away and leaving only the fused protein on the polynucleotide strand.

Alternatively, the esterase may be added separately instead of being fused to the polymerase. In this case, the residual non-incorporated nucleotides may be washed away and an esterase may then be added to the reaction to cleave the redox label at low pH and low temperature as described for the esterase EstA8.

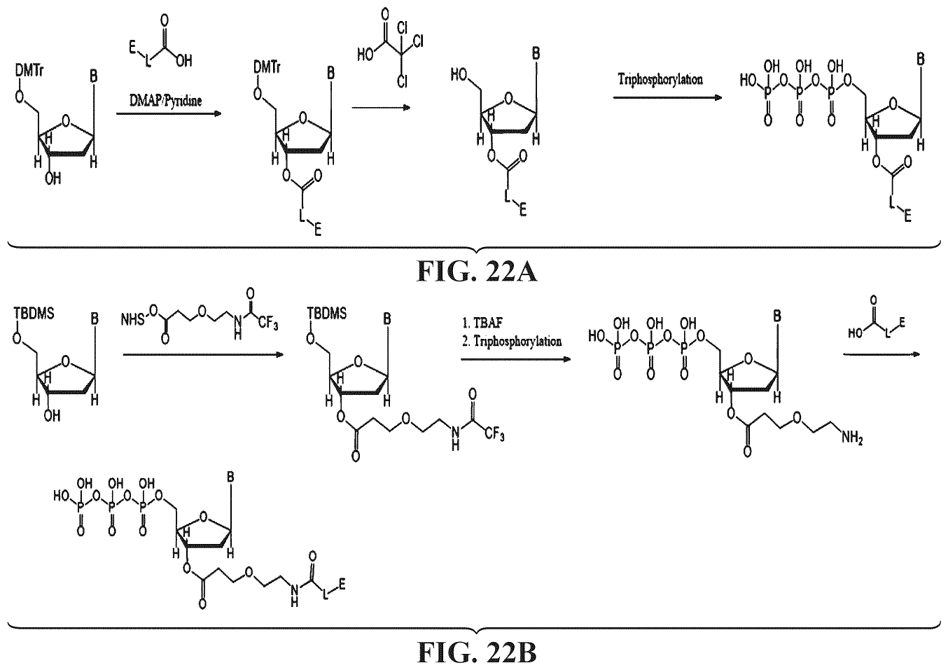

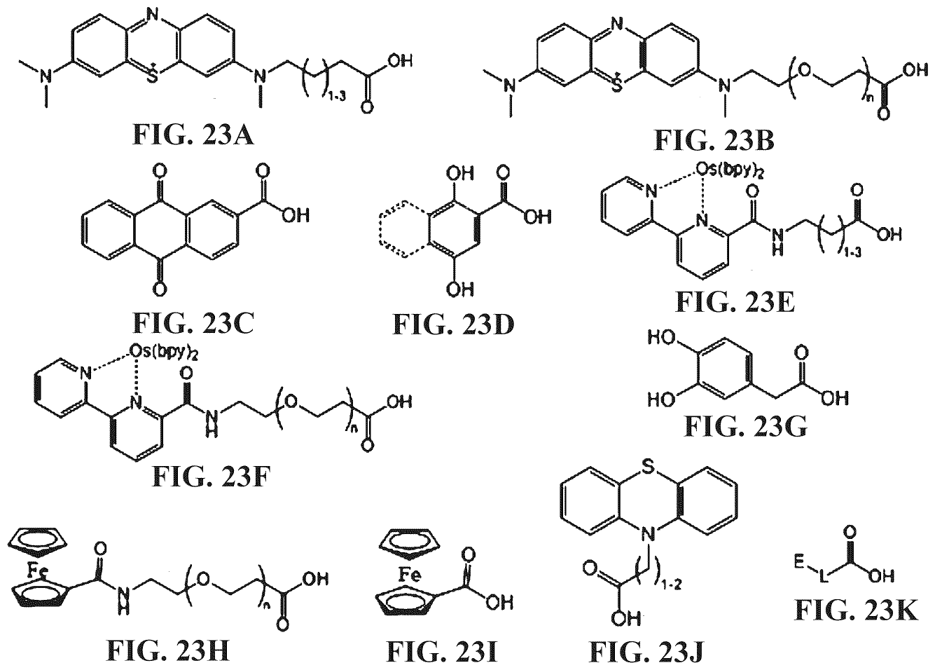

FIGS. 22A and 22B illustrate example routes for synthesis of dNTPs modified with electroactive labels attached at the 3′-OH position via an ester group. FIGS. 23A-23J illustrate example electroactive labels with ester functional groups. FIG. 23K illustrates a general formula for an electroactive label with a linker and an ester functional group, where E refers to the electroactive label, and L refers to the linker.

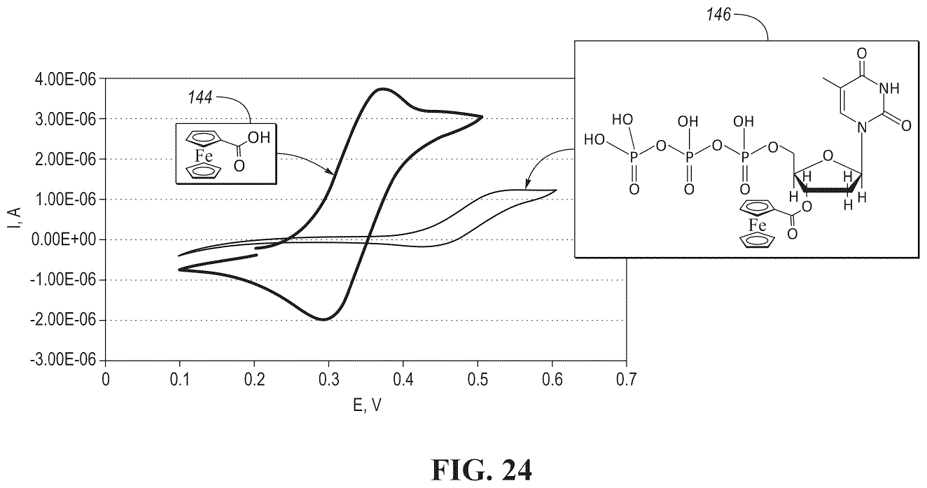

Some electroactive labels may exhibit different redox behavior depending on the form in which they exist. For example, an electroactive label may exhibit a particular redox behavior when it is in the form of a free acid, and a different redox behavior when it is in the form of an ester conjugated to the nucleotide. Non-limiting examples of such labels are Ferrocene carboxylic acid, anthraquinone-2-carboxylic acid, 10-phenothiazine-2-carboxylic acid, and phenazine-2-carboxylic acid. FIG. 24 illustrates cyclic voltammograms of free ferrocene carboxylic acid 144 and Compound C3 146 (a ferrocene-labeled dTTP reversible terminator with an ester group and no linker). The two forms of this ferrocene label may be easily distinguished based on their unique electrochemical signals. Additionally, these detectable differences in redox behavior further allow for the specific detection of electroactive labels released by the action of a polymerase thereby indicating an incorporation event. In this way, if the voltages applied to the nanogap sensor electrodes only produce electrochemical interactions with the free form, only free labels will be detected by the sensor. This result further serves to lower the background signal from labeled dNTPs present in the bulk solution as labels bound to dNTPs will not be detected under these conditions.

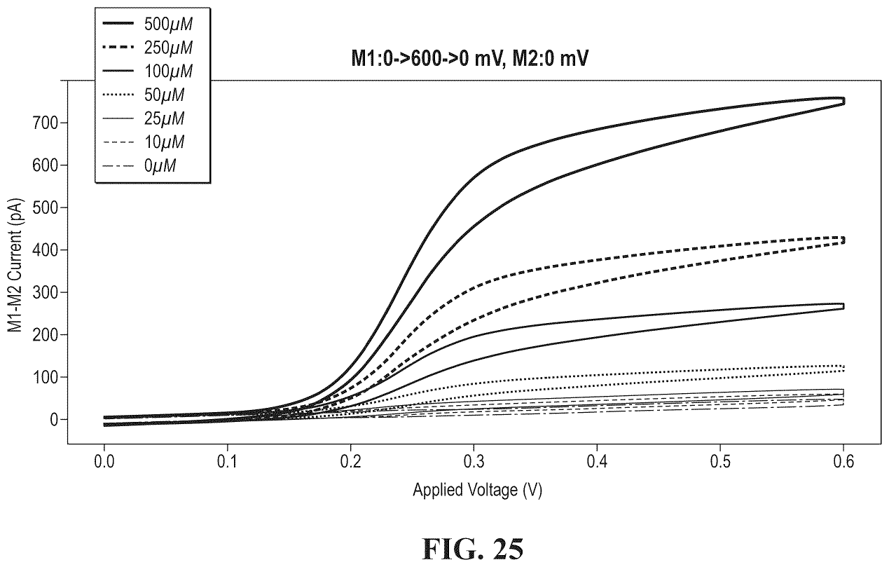

FIG. 25 illustrates measurements of a ferrocene carboxylic acid signal at different concentrations. The voltage on first electrode was kept constant at 0 mV, while the voltage on second electrode was swept between 0 and 600 mV.

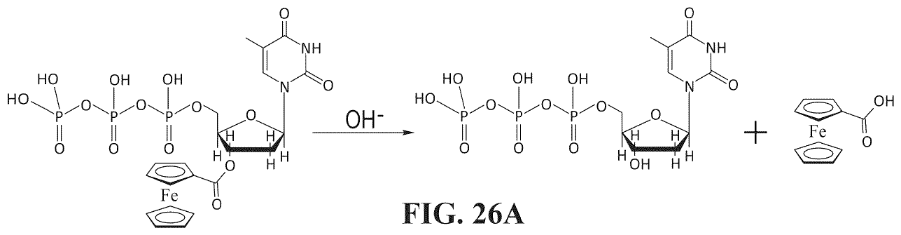

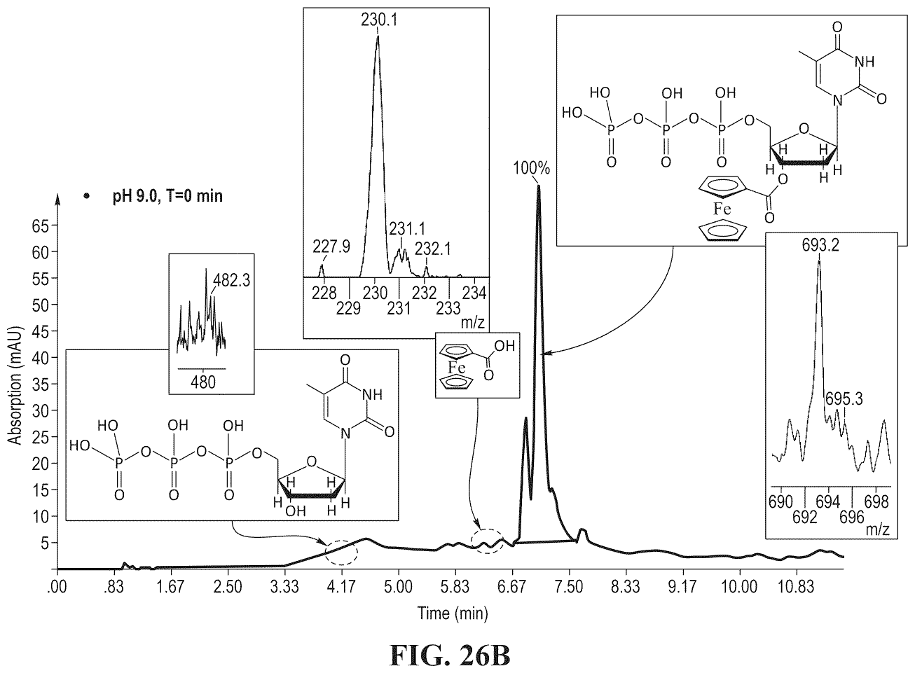

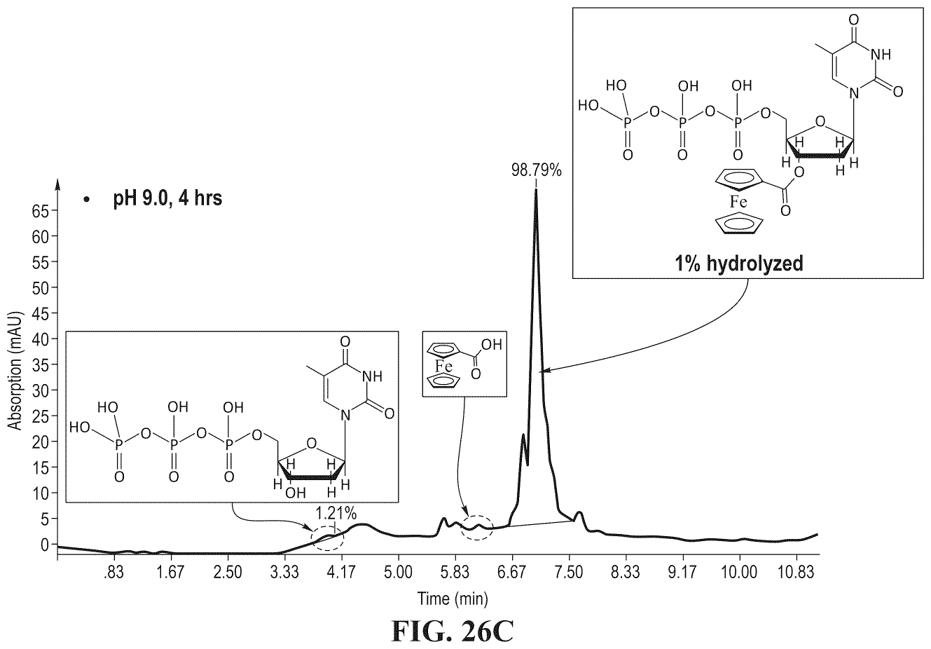

FIGS. 26A-C demonstrates that dTTP-Fc is stable against spontaneous hydrolysis at pH 9. Spontaneous hydrolysis was measured at 0 minutes and at 4 hours. Only 1% hydrolysis had occurred at 4 hours showing that dTTP-Fc is stable.

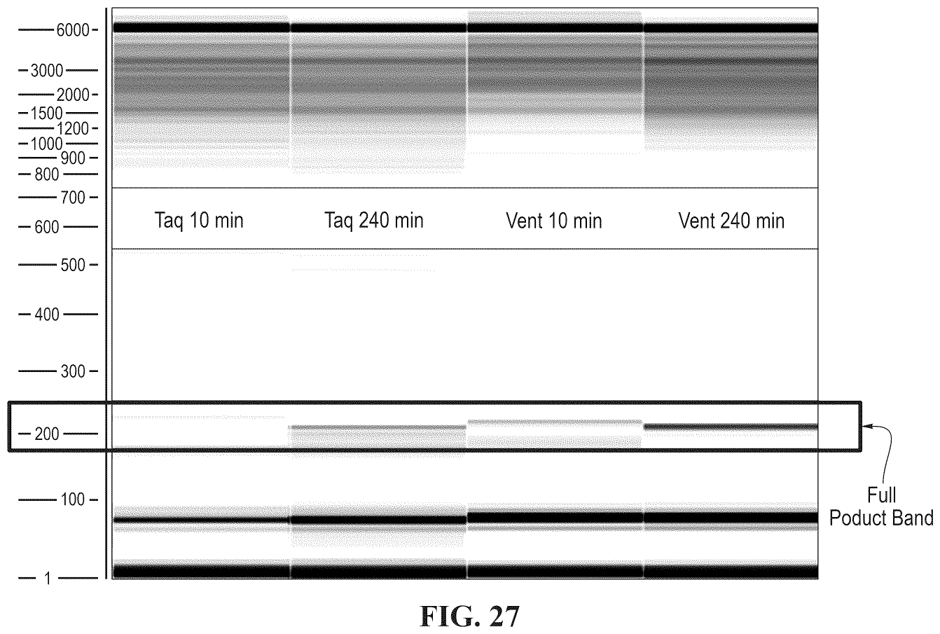

FIG. 27 illustrates successful incorporation of dTTP-Fc into DNA by Vent and Taq polymerases at pH 8.8. Bands for the full-length product are visible in the lanes containing samples of DNA produced at 240 minutes by both Taq and Vent polymerases. This experiment was performed at pH 8.8. Formation of a full-length product indicates that the ester group is cleaved without any additional external stimulus. The action of the enzyme under these experimental conditions is sufficient to release ferrocene carboxylic acid label and enable incorporation of the next nucleotide.

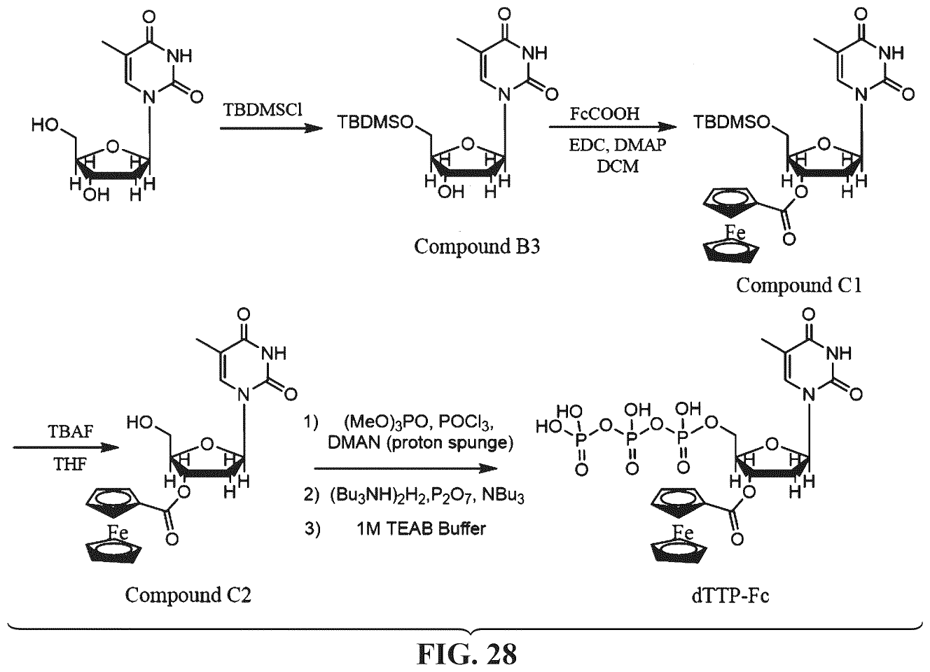

FIG. 28 illustrates a method for synthesizing compound C3 146 which is a ferrocene-labeled dTTP reversible terminator with an ester group and no linker.

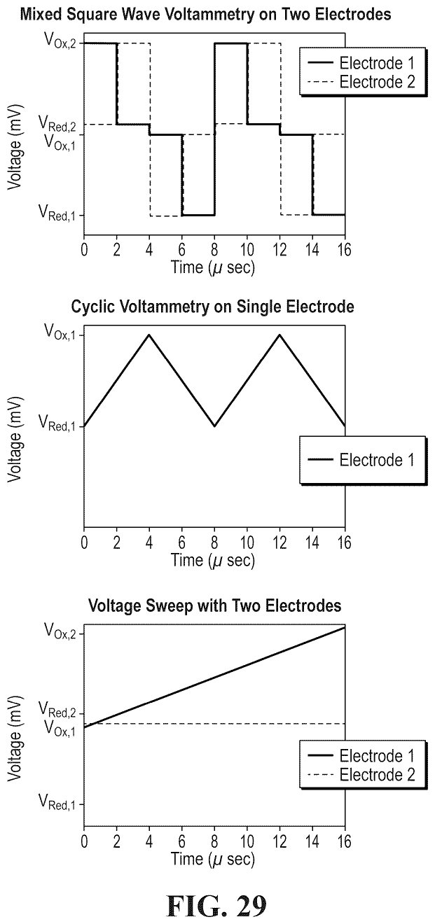

FIG. 29 illustrates potential voltammetry techniques that may be utilized with the systems and methods described herein.

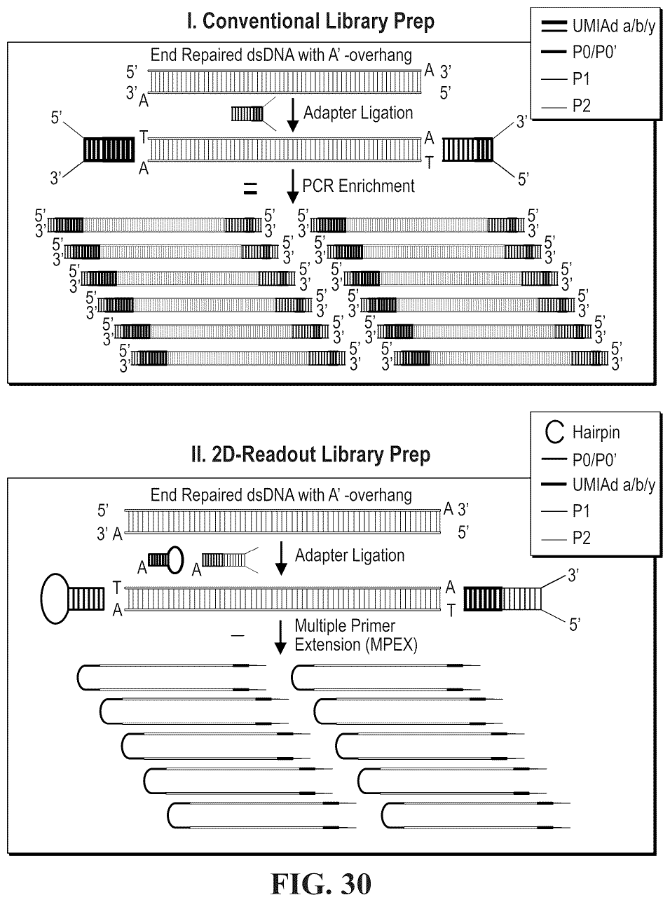

According to various embodiments, conventional targeted or universal library preparation methods may be utilized where the amplicons carry known flanking sequences representing universal primer annealing sites for the sequencing-by-synthesis reaction. Template DNA may exemplarily be prepared according to one of the following two library preparation strategies as described in FIG. 30.

Conventional library prep: DNA may be blunt end repaired and dA-tailed to be ligated with dT-tailed pseudo-double-stranded, Y-shaped adapters and amplified via PCR to gain asymmetric flanking sites. Amplicon sizes may range from 50-5000 bp. To ensure homogenous cluster formation, the DNA can be denatured prior to loading on the electronic nanosensor. Adapters may contain unique molecular identifiers to overcome PCR-introduced errors and to allow unique and full DNA reconstruction where a two redox-label readout is preferred during sequencing. Three and four redox-label readout strategies may also be compatible with this library preparation strategy. In the figure key, UMI Ad a/b/y refers to a UMIAdapter 1-n (unique molecular identifier) of >40 bp. It unambiguously pairs and aligns the sense and antisense strand. It is required for error correction through consensus calling. The number of UMIs is greater than the number of different molecules. P0/P0′ is a recognition site for primer extension during adapter generation. It is around 20 bp. P1 and P2 are asymmetric sequence part 1 and part 2 respectively. They are around 20 bp. They are recognition sites for PCR and primer extension during redox labeling. They serve as an internal control for orientation. They mark the start and end of a molecule and give the location for the UMI on the sense and antisense strands.

2D-readout library prep: DNA may be blunt end repaired and dA-tailed to be ligated with dT-tailed pseudo-double-stranded, Y-shaped adapters and hairpin adapters. A positive selection for asymmetrically ligated products may follow. These asymmetrically ligated products may be used for the sequencing reaction directly. Alternatively, an amplification may be performed via multiple primer extension or other isothermal amplification method including but not limited to LAMP, RPA, rITA, etc. Product/amplicon size may be determined by the processivity of the polymerases that are used throughout the process. Adapters may contain unique molecular identifiers to overcome amplification-introduced errors. To ensure homogenous cluster formation, the DNA may be denatured prior to loading on the electronic nanosensor. The linkage of sense and antisense strand may increase the accuracy of the sequencing readout. In the figure key, HairpinAdapter refers to a covalent linker between the sense and antisense strand. P0/P0′ is a recognition site for sense/antisense switch in hairpin adapter. They are around 20 bp. UMI Ad a/b/y refers to a UMIAdapter 1-n (unique molecular identifier) of >40 bp. It unambiguously pairs and aligns the sense and antisense strand. It is required for error correction through consensus calling. The number of UMIs is greater than the number of different molecules. P1 and P2 are asymmetric sequence part 1 and part 2 respectively. They are around 20 bp. They are recognition sites for PCR and primer extension during redox labeling. They serve as an internal control for orientation. They mark the start and end of a molecule and give the location for the UMI on the sense and antisense strands.

Alternatively, a given DNA sample may also be directly sequenced without prior library preparation by using target specific sequencing primers. The primer annealing to the prepared DNA may occur on the chip (e.g., denaturation for 1 min at 95° C. and annealing for 1 min at 60° C.) provided that the polymerase is capable of withstanding denaturing conditions. More favorably, the primer annealing may occur prior to loading onto the chip while the primer-template DNA conjugate may remain stable until it reaches the polymerase.

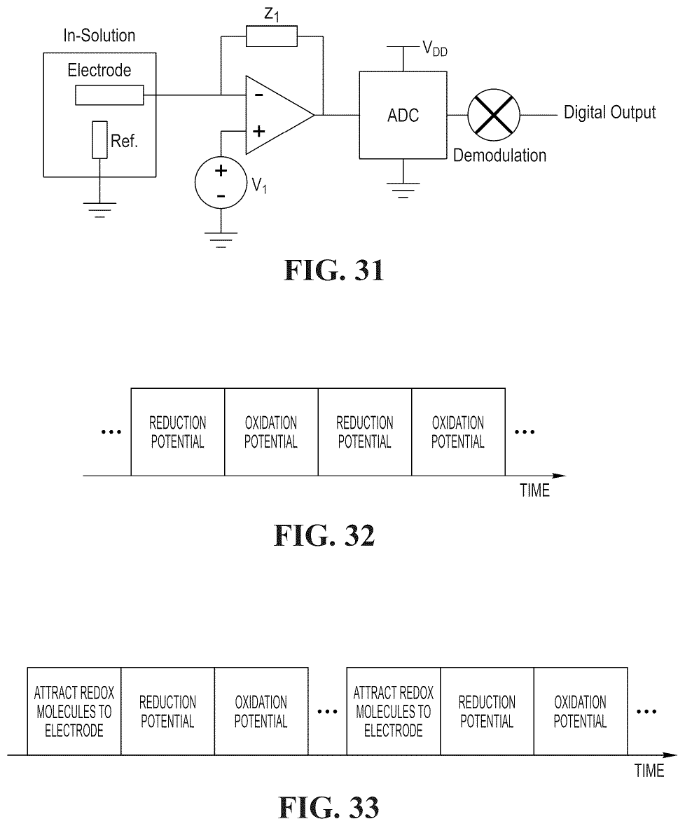

According to at least an embodiment, cleaved redox molecules may also be sensed using an electronic nanosensor having only one electrode. (See the electronic nanosensor 120 in FIGS. 2B and 2C). FIG. 31 illustrates the concept for the readout circuit. With this circuit it is possible to bias the electrode in solution at a potential which is determined by the voltage source V1. If electrons transfer from the electrode to the electroactive label (also referred to as a redox molecule), a current flows via the feedback impedance Z1 which results in a voltage drop across the impedance Z1. This voltage drop can be processed either in the analog or digital domain. In this example the voltage is converted into a digital signal by the Analog-to-Digital Converter (ADC). The voltage V1 and therefore the potential on the electrode can be changed over time in a way so that a time-variant signal at the output of this circuit can be measured. This signal is proportional to the number of redox molecules close to the surface of the electrode. FIG. 32 illustrates the different phases for applying potentials to the electrode. There are two kinds of phases that the circuit toggles between. If the reduction potential is applied the redox molecules get reduced and if the oxidation potential is applied the redox molecules get oxidized. This results in an alternating current through the feedback impedance Z1 and therefore in an alternating signal at the input of the ADC. The signal at the output of the ADC gets multiplied with a positive or negative value, depending on what phase the circuit is in. This is equal to a demodulation of the signal. Demodulation of the signal involves extracting information from the transmitted signal. One way to implement demodulation is as follows. Whenever the circuit is in the reduction phase, the output signal of the ADC is multiplied with a positive value and whenever the circuit is in the oxidation phase the output of the ADC is multiplied with a negative value.

In another example of sensing cleaved redox molecules using an electronic nanosensor with only one electrode, the cleaved redox molecules may be attracted to the sensing electrode. This can be done electrostatically. FIG. 33 illustrates the different phases over time. In addition to the alternating reduction and oxidation phases, a phase is added to attract redox molecules to the electrode surface. The sequence of attracting, reducing and oxidizing redox molecules may then be repeated over and over.

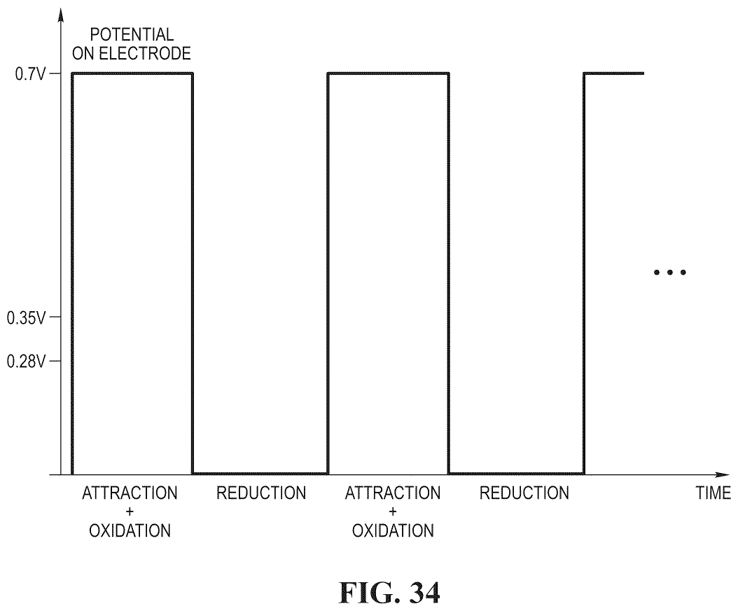

The phase of attracting redox molecules electrostatically to the electrode surface may also be integrated with either the oxidation or the reduction phase. This is shown as an example in FIG. 34 for a Ferrocene electroactive label. Cleaved Ferrocene in solution has an oxidation potential of 0.35V and a reduction potential of 0.28V. The voltage on the electrode oscillates between a voltage below the reduction potential, in this case 0V, and a voltage above the oxidation potential, in this case 0.7V. Freshly cleaved Ferrocene is in reduced form and charged negatively. The voltage of 0.7V on the electrode will electrostatically attract Ferrocene and once Ferrocene moves close enough to the electrode it will be oxidized and an electron will be transferred to the electrode. In the next phase Ferrocene is reduced and in the next phase it will be oxidized again. Whenever the oxidizing potential is applied, Ferrocene will be electrostatically attracted to the electrode and whenever the reduction potential is applied, there will be no electrostatic force on Ferrocene. Over time Ferrocene sees in average an electrostatic force which is attractive.

### EXAMPLES

### Example 1: Synthesis of a Ferrocene-Labeled dTTP Reversible Terminator with Ester Group (dTTP-Fc)

Example compound dTTP-Fc was synthesized using steps described in FIG. 28. Thymidine (2 g, 8.2 mmol) and imidazole (1.1 g, 16.5 mmol) were dissolved in 12 mL of dry DMF under inert atmosphere. TBDMSCl (1.3 g, 8.7 mmol) was added portion-wise. The reaction was kept at room temperature and under inert atmosphere overnight, then concentrated on rotavap. The product (B3) was isolated using silica gel column with chloroform/MeOH linear gradient (0-50% MeOH). Compound B3 was obtained as white solid (1.55 g, 53% yield).

Compound B3 (1.5 g, 4.3 mmol), Ferrocene carboxylic acid (0.5 g, 2.1 mmol), EDC (0.46 g, 2.4 mmol), and DMAP (13.3 mg, 0.1 mmol) were dissolved in 50 mL of dry DCM in oxygen-free atmosphere and stirred at room temperature. Conversion of compound B3 was monitored by TLC using Hexane/Ethyl Acetate as eluent. After the reaction was completed, the solvents were evaporated and the product was purified using silica gel flash chromatography with Hexane/Ethyl Acetate linear gradient. The yield of the isolated product C1: 0.866 g (56%).

The obtained product C1 (0.38 g) was redissolved in 15 mL anhydrous THF. 1.0 M tetrabutyl ammonium fluoride (TBAF) (1.5 mL) in THF was added dropwise to the above solution and kept stirring for 30 minutes. After the reaction was completed as indicated from TLC, the solvent was evaporated, followed by addition of 20 mL saturated NaHCO₃ solution. The solution was extracted with 2×20 mL ethyl acetate. The organic layer was combined and dried using anhydrous MgSO₄. The solution was filtered, and the solvent was removed to obtain compound C2 as a yellow viscous liquid (0.3 g, yield 100%).

The final compound dTTP-Fc was obtained after a triphosphorylation procedure: Measure compound C2 (50 mg, 0.11 mmol) and 1,8-bis(dimethylamino)naphthalene (DMAN) (23 mg, 0.11 mmol) in a 50 mL round bottom flask and take out tributylammonium pyrophosphate (TBAP) (90 mg, 0.16 mmol) in a separate vial. Keep both for drying under high vacuum over P₂O₅ (ca 500 mg) for next 1 h. Chill Tributyl amine (NBu3) (0.16 mL, 0.66 mmol) to −20° C. After the drying process, perform a vacuum-nitrogen cycle 3 times for both the flask and the vial. Add 1 mL of acetonitrile into the vial containing TBAP and chill to −20° C. Then, slowly add 1 mL of trimethyl phosphate to the flask containing C2 and DMAN, and stir the flask continuously for 10 minutes under a mix of ice and dry ice bath. Then add phosphoryl chloride (0.027 mL, 0.3 mmol) dropwise to the flask over a period of 5 minutes and keep stirring for another 30 minutes under ice/dry ice bath. Then add chilled NBu3 and TBAP into the reaction mixture and stir for the next 1 h under ice cold conditions. Introduce 1.0 M chilled TEAB Buffer to the reaction mixture and stir for another 1 h. Reduce the solution under vacuum to remove any remaining organic solvent. Freeze the obtained aqueous solution of the reaction mixture at −80° C. for lyophilization. The frozen mixture was lyophilized and purified using reverse-phase HPLC (C-18 column, TEAB/Acetonitrile linear gradient) to afford dTTP-Fc as a white solid (8.2 mg).

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A method for nucleic acid sequencing, comprising:
providing at least one device comprising an electronic nanosensor;
providing a sample including a fragmented polynucleotide strand to the at least one device;
clonally amplifying the fragmented polynucleotide strand within the at least one device to produce a clonally amplified cluster;
exposing the clonally amplified cluster to a reaction solution comprising a polymerase enzyme capable of incorporating a nucleotide modified with an electroactive label covalently bound to the 3′-OH group of a sugar ring of the nucleotide into a polynucleotide strand, and at least one nucleotide modified with an electroactive label covalently bound to the 3′-OH group of a sugar ring of the nucleotide so that the at least one nucleotide modified with an electroactive label covalently bound to the 3′-OH group of a sugar ring of the nucleotide is incorporated into the polynucleotide strand;
cleaving the electroactive label from the incorporated nucleotide so that the electroactive label diffuses toward the electronic nanosensor; and
detecting a signal produced when the electroactive label is present within a sensing zone of the electronic nanosensor.

2. The method of claim 1, further comprising measuring a first electrochemical signal at the nanosensor when the electroactive label is covalently bound to the 3′-OH of a sugar ring of the nucleotide and measuring a second electrochemical signal at the nanosensor after the electroactive label is released from the nucleotide, wherein the second electrochemical signal is measurably distinguishable from the first electrochemical signal.

3. The method of claim 1, wherein the signal produced when the electroactive label is present within the sensing zone of the electronic nanosensor is the current (I) as a function of applied potential (V).

4. The method of claim 1, wherein the electroactive label on the incorporated nucleotide is cleaved from the nucleotide by the polymerase enzyme.

5. The method of claim 1, further comprising applying a stimulus to induce cleavage of the electroactive label on the incorporated nucleotide to expose the 3′-OH end of the growing polynucleotide strand to allow for subsequent addition of the next nucleotide.

6. The method of claim 5, wherein the stimulus is a chemical reagent, and wherein the chemical reagent is added to the system after each nucleotide incorporation.

7. The method of claim 5, wherein the stimulus is the activity of an enzyme.

8. The method of claim 5, wherein the stimulus is an external trigger.

9. The method of claim 8, wherein the external trigger comprises an electrochemically induced change in pH of the reaction solution in the area of the polynucleotide strand.

10. The method of claim 8, wherein the external trigger comprises an electrochemically induced change in pH and in temperature of the reaction solution in the area of the polynucleotide strand.

11. A system for nucleic acid sequencing comprising:
at least one device including:
at least one electronic nanosensor; and
a controller configured to:
provide a signal to the at least one device to promote delivery to the at least one device of a sample including a fragmented polynucleotide strand, and a reaction solution including a polymerase enzyme capable of incorporating a nucleotide modified with an electroactive label covalently bound to the 3′-OH group of a sugar ring of the nucleotide into a polynucleotide strand, and at least one nucleotide modified with an electroactive label covalently bound to the 3′-OH group of a sugar ring of the nucleotide;
provide a signal to the at least one device to promote clonal amplification of the polynucleotide strand and to promote incorporation of the at least one nucleotide modified with an electroactive label covalently bound to the 3′-OH group of a sugar ring of the nucleotide into the amplified polynucleotide strands; and
apply a voltage to at least one electrode of the electronic nanosensor; and
measure the current (I) as a function of applied potential (V) when an electroactive label that has been cleaved from the incorporated nucleotide is present within the sensing zone of the electronic nanosensor.

12. The system of claim 11, wherein the device includes an array of electronic nanosensors.

13. The system of claim 11, wherein the electroactive label on the incorporated nucleotide is cleaved from the nucleotide by the polymerase enzyme.

14. The system of claim 11, wherein the controller is further configured to apply a stimulus to the at least one device that induces cleavage of the electroactive label from the incorporated nucleotide so that the electroactive label diffuses toward the electronic nanosensor;

15. The system of claim 14, wherein the stimulus is an external trigger.

16. The system of claim 15, wherein the external trigger comprises an electrochemically induced change in pH of the reaction solution in an area of the polynucleotide strand.

17. A method for nucleic acid sequencing, comprising:
providing at least one device comprising an electronic sensor having a sensing electrode;
providing a sample including a fragmented polynucleotide strand to the at least one device;
clonally amplifying the fragmented polynucleotide strand within the at least one device to produce a clonally amplified cluster;
exposing the clonally amplified cluster to a reaction solution comprising a polymerase enzyme capable of incorporating a nucleotide modified with an electroactive label covalently bound to the 3′-OH group of a sugar ring of the nucleotide into a polynucleotide strand, and at least one nucleotide modified with an electroactive label covalently bound to the 3′-OH group of a sugar ring of the nucleotide so that the at least one nucleotide modified with an electroactive label covalently bound to the 3′-OH group of a sugar ring of the nucleotide is incorporated into the polynucleotide strand;
cleaving the electroactive label from the incorporated nucleotide so that the electroactive label diffuses toward the electronic sensor;
applying a potential on the sensing electrode, wherein the potential oscillates between a reduction potential and an oxidation potential of the electroactive label; and
detecting a signal transmitted with the oxidation and/or reduction of the cleaved electroactive label.

18. The method of claim 17, further comprising demodulating the transmitted signal in phase with the applied potential.

19. The method of claim 17, further comprising applying a potential to the sensing electrode to electrostatically attract the electroactive labels to the sensing electrode.

20. The method of claim 17, further comprising applying a stimulus to induce cleavage of the electroactive label on the incorporated nucleotide.
